(19) United States
(12) Patent Application Publication
(10) Pub. No.: US 2026/0042800 A1
(43) Pub. Date: Feb. 12, 2026

(54) ANTIMICROBIAL PEPTIDOMIMETICS

(71) Applicants: Basilea Pharmaceutica International AG, Allschwil, Allschwil (CH); Universität Zürich, Zürich (CH)

(72) Inventors: Emile Brabet, Allschwil (CH); Mohammed Benghezal, Allschwil (CH); Grégory Upert, Allschwil (CH); Françoise Jung, Allschwil (CH); Carolin D’Arco, Allschwil (CH); Sarah Stiegeler, Allschwil (CH)

(21) Appl. No.: 19/290,517

(22) Filed: Aug. 5, 2025

Related U.S. Application Data
(60) Provisional application No. 63/679,696, filed on Aug. 6, 2024.

(30) Foreign Application Priority Data
Aug. 6, 2024 (EP) 24193117.9
Apr. 8, 2025 (EP) 25169131.7

(51) Int. Cl. C07K 7/08; A61K 38/00; A61P 31/04

(52) U.S. Cl. CPC C07K 7/08; A61P 31/04; A61K 38/00

(57) ABSTRACT

The present invention is directed to peptidomimetics having antibacterial activity, especially against Gram-negative bacteria. The peptidomimetics of the invention are compounds of the general formula (I),

P¹-P²-P³-P⁴-P⁵-P⁶-P⁷-P⁸-P⁹-P¹⁰-P¹¹-P¹²-P¹³-P¹⁴-P¹⁵-P¹⁶
(I)

and salts thereof, in particular pharmaceutically acceptable salts thereof, as described in the description and in the claims. The invention is also directed to therapeutic uses of the peptidomimetics for the treatment or prevention of bacterial infections and diseases related to bacterial infections and to non-therapeutic uses of the peptidomimetics for preserving or disinfecting foodstuffs, cosmetics, medicaments or other nutrient-containing materials.

### CROSS REFERENCE TO RELATED APPLICATIONS

This application claims priority from U.S. Provisional Application 63/679,696 filed on Aug. 6, 2024, European Patent Application No. 24193117.9 filed on Aug. 6, 2024 and European Patent Application No. 25169131.7 filed Apr. 8, 2025. The priority of said US Provisional Application and European Patent Applications are claimed. Each of the prior mentioned applications is hereby incorporated by reference herein in its entirety.

### STATEMENT REGARDING FEDERALLY SPONSORED RESEARCH OR DEVELOPMENT

This invention was made with U.S. Government support under Agreement Number 75A50122C00028, awarded by the U.S. Department of Health and Human Services. The U.S. Government has certain rights in the invention.

### REFERENCE TO AN ELECTRONIC SEQUENCE LISTING

The instant application contains a Sequence Listing which has been submitted electronically in XML file format and is hereby incorporated by reference in its entirety. Said XML copy, created on Aug. 4, 2025, is named 104610-111514_P1175US01_SL.xml and is 77,214 bytes in size.

### FIELD OF THE INVENTION

The present invention is directed to peptidomimetics having antimicrobial activity, especially against Gram-negative bacteria. The peptidomimetics of the invention are compounds of the general formula (I),

(I)
p¹_p²_p³_p⁴_p⁵_p⁶_p⁷_p⁸_p⁹_p¹⁰_p¹¹_p¹²_p¹³_p¹⁴_p¹⁵_p¹⁶

and pharmaceutically acceptable salts thereof, as described herein below. The invention is also directed to therapeutic uses of the peptidomimetics for the treatment or prevention of bacterial infections and diseases related to bacterial infections and to non-therapeutic uses of the peptidomimetics for preserving or disinfecting foodstuffs, cosmetics, medicaments or other nutrient-containing materials.

There are limited treatment options for carbapenem-resistant Enterobacteriaceae (CRE) infections. Antibiotics that more frequently show in vitro activity against CRE include colistin, tigecycline and fosfomycin. However, the data on their effectiveness and clinical experience is limited. There are also more frequent adverse effects, rapid development of resistance during treatment, and increasing resistance globally. Colistin is frequently being used to treat CRE infections, but colistin resistance may develop in CRE-infected patients treated with colistin. Since 2015, the discovery of transferable plasmid-mediated colistin resistance genes (mcr 1-5) that can transmit colistin resistance more easily between bacteria has further increased the risk of colistin resistance spreading (Giamarellou H. et al., Antimicrob Agents Chemother. 2013, 57 (5), 2388-90).

None of the recently approved antibiotics or those in late-stage development have a satisfactory coverage of CRE. Notably, new beta-lactam combinations lack activity against metallo-beta-lactamase (MBL) producing organisms. Ceftazidime/Avibactam (CAZ-AVI), most commonly used novel antibiotic against CREs is not active against MBL organisms. Furthermore, reports of CAZ-AVI-resistant CRE strains that have developed resistance during treatment with CAZ-AVI, alone or in combination with other antibiotics, soon after the launch of CAZ-AVI. After these reports of concern, ECDC has issued a rapid risk assessment report regarding this issue in Jun. 12, 2018. The new aminoglycoside plazomicin has safety warnings (nephrotoxicity, ototoxicity, neuromuscular blockade and fetal harm) in the prescribing information.

Thus, there is an on-going need for the development of antibiotics that can be used for the effective treatment of CRE infections.

The natural antimicrobial peptide thanatin, a 21-residue inducible insect defense peptide (Fehlbaum P. et al., Proc. Natl. Acad. Sci. USA 1996, 93, 1221-1225), is targeting the lipopolysaccharide transport protein LptA of Gram-negative bacteria, which leads to inhibition of LPS transport and outer membrane (OM) biogenesis (Vetterli S. U. et al., Sci. Adv. 2018; 4: eaau2634). Thanatin is active against carbapenem-resistant Enterobacteriaceae including pan resistant strains. These highly resistant organisms can cause a variety of infections including complicated urinary tract infections (cUTI), complicated intra-abdominal infections (cIAI), hospital- or ventilator-associated pneumonia (HAP/VAP), or bloodstream infections (BSI).

WO 2022/028737, WO 2022/028738, WO 2023/012319 and Schuster et al Sci. Adv. 2023, 9, eadg3683 describe thanatin-derived peptidomimetics having 16, 17, 18 or 19 amino acid or amino acid derived residues having antibacterial activity, especially against Gram-negative bacteria.

The present invention provides novel peptidomimetics having 16 amino acid or amino acid derived residues.

In a first aspect, the invention provides a compound of the general formula (I),

(I)
(SEQ ID NO: 17)
p¹_p²_p³_p⁴_p⁵_p⁶_p⁷_p⁸_p⁹_p¹⁰_p¹¹_p¹²_p¹³_p¹⁴_p¹⁵_p¹⁶

wherein
P¹ is Gua-Val;
P² is Pro, Pro(4R)OMe, Pro(3,4dehydro), Pic, Pro(4R)NH₂), Ndab, NalloThr, or Hyp;
P³ is Ile;
P⁴ is Val(3OH);
P⁵ is Tyr;
P⁶ is Cys or Pen;
P⁷ is Asn, Leu, Ile, Ser, Dap, or His;
P⁸ is Arg;
P⁹ is Arg, Lys, Dab, Dab(iPr), Pro or Hyp;
P¹⁰ is Ser or Thr;
P¹¹ is ^{D}Dab, ^{D}Dab(iPr), or ^{D}Arg;
P¹² is Lys, Ile, Ser, Dab, Orn, or Cit;
P¹³ is Cys or Pen;
P¹⁴ is Dab, Dab(iPr), Lys, Gln, Ser, or Tyr;
P¹⁵ is Arg, Dab, Orn, Orn (iPr), Ser, or Thr;
P¹⁶ is Nle, Cha, or Tyr;
or a salt thereof,
wherein Cys or Pen at P⁶ and Cys or Pen at P¹³ optionally form a disulfide bridge between P⁶ and P¹³;
with the proviso that at least three amino acid residues among the four amino acid residues at positions P⁹, P¹², P¹⁴ and P¹⁵ are basic amino acid residues selected from Dab, Lys, Arg, or Dab(iPr) at P⁹, Lys, Orn, or Dab at P¹², Lys, Dab or Dab(iPr) at P¹⁴ and Arg, Dab, Orn, or Orn (iPr) at P¹⁵.

In a further aspect, the invention provides a pharmaceutical composition containing a compound of formula (I) or a pharmaceutically acceptable salt thereof or a mixture of compounds of formula (I) or pharmaceutically acceptable salts thereof and at least one pharmaceutically inert carrier.

In a further aspect, the invention provides a compound of formula (I) or a salt thereof for use as a pharmaceutically active substance having antibiotic activity.

In a further aspect, the invention provides use of a compound of formula (I) or a salt thereof as a pharmaceutically active substance having antibiotic activity.

In a further aspect, the invention provides a compound of formula (I) or a pharmaceutically acceptable salt thereof for use as a medicament.

In a further aspect, the invention provides a compound of formula (I) or a pharmaceutically acceptable salt thereof for use in a method of treating or preventing a bacterial infection or a disease related to such infection.

In a further aspect, the invention provides use of a compound of formula (I) or a pharmaceutically acceptable salt thereof in the manufacture of a medicament for the treatment or prevention of a bacterial infection or a disease related to such infection.

In a further aspect the invention provides a method of treating or preventing a bacterial infection or a disease related to such infection in a subject in need thereof, comprising administering a therapeutically effective amount of a compound of formula (I) or a pharmaceutically acceptable salt thereof to the subject.

In a further aspect the invention provides use of a compound for formula (I) or a salt thereof as a disinfectant or preservative for foodstuffs, cosmetics, medicaments, and/or other nutrient-containing materials.

In some embodiments, the compound of formula (I) is a compound, wherein
P¹ is Gua-Val;
P² is Pro, Pro(4R)OMe, Pro(3,4dehydro), Pic, Pro((4R)NH₂), Ndab, NalloThr, or Hyp;
P³ is Ile;
P⁴ is Val(3OH);
P⁵ is Tyr;
P⁶ is Cys or Pen;
P⁷ is Asn, Leu, Ile, Ser, Dap, or His;
P⁸ is Arg;
P⁹ is Arg, Lys, Dab, Dab(iPr), Pro or Hyp;
P¹⁰ is Ser or Thr;
P¹¹ is ^{D}Dab, ^{D}Dab(iPr), or ^{D}Arg;
P¹² is Dab;
P¹³ is Cys or Pen;
P¹⁴ is Dab, Dab(iPr), Lys, Gln, Ser, or Tyr;
P¹⁵ is Arg, Dab, Orn, Orn (iPr), Ser, or Thr;
P¹⁶ is Nle, Cha, or Tyr;
or a salt thereof,
wherein Cys or Pen at P⁶ and Cys or Pen at P¹³ optionally form a disulfide bridge between P⁶ and P¹³;
with the proviso that at least two amino acid residues among the three amino acid residues at positions P⁹, P¹⁴ and P¹⁵ are basic amino acid residues selected from Dab, Lys, Arg, or Dab(iPr) at P⁹, Lys, Dab or Dab(iPr) at P¹⁴ and Arg, Dab, Orn, or Orn (iPr) at P¹⁵.

In some embodiments, the compound of formula (I) is a compound, wherein
P¹ is Gua-Val;
P² is Pro, Pro(4R)OMe, Pro((4R)NH₂), or Hyp;
P³ is Ile;
P⁴ is Val(3OH);
P⁵ is Tyr;
P⁶ is Cys or Pen;
P⁷ is Asn;
P⁸ is Arg;
P⁹ is Lys, Dab, Dab(iPr), Pro or Hyp;
P¹⁰ is Ser or Thr;
P¹¹ is ^{D}Dab, ^{D}Dab(iPr);
P¹² is Dab;
P¹³ is Cys or Pen;
P¹⁴ is Dab, Dab(iPr) or Lys;
P¹⁵ is Arg, Dab, Orn, Orn (iPr), Ser, or Thr;
P¹⁶ is Tyr;
or a salt thereof,
wherein Cys or Pen at P⁶ and Cys or Pen at P¹³ optionally form a disulfide bridge between P⁶ and P¹³;
with the proviso that at least one amino acid residue among the two amino acid residues at positions P⁹ and P¹⁵ are basic amino acid residues selected from Dab, Lys or Dab(iPr) at P⁹, and Arg, Dab, Orn, or Orn (iPr) at P¹⁵.

In some embodiments, the compound of formula (I) is a compound, wherein
P¹ is Gua-Val;
P² is Hyp;
P³ is Ile;
P⁴ is Val(3OH);
P⁵ is Tyr;
P⁶ is Pen;
P⁷ is Asn or Ser;
P⁸ is Arg;
P⁹ is Dab, Pro or Hyp;
P¹⁰ is Ser or Thr;
P¹¹ is ^{D}Dab;
P¹² is Lys or Dab;
P¹³ is Cys;
P¹⁴ is Dab;
P¹⁵ is Arg, Dab or Thr;
P¹⁶ is Tyr;
or a salt thereof,
wherein Pen at P⁶ and Cys at P¹³ optionally form a disulfide bridge between P⁶ and P¹³;
with the proviso that at least one amino acid residue among the two amino acid residues at positions P⁹ and P¹⁵ are basic amino acid residues selected from Dab at P⁹, and Arg and Dab at P¹⁵.

In some embodiments, the compound of formula (I) is a compound, wherein
P¹ is Gua-Val;
P² is Hyp;
P³ is Ile;
P⁴ is Val(3OH);
P⁵ is Tyr;
P⁶ is Pen;
P⁷ is Asn;
P⁸ is Arg;
P⁹ is Dab, Pro or Hyp;
P¹⁰ is Ser or Thr;
P¹¹ is ^{D}Dab;
P¹² is Dab;
P¹³ is Cys;
P¹⁴ is Dab;
P¹⁵ is Dab or Thr;
P¹⁶ is Tyr;
or a salt thereof,
wherein Pen at P⁶ and Cys at P¹³ optionally form a disulfide bridge between P⁶ and P¹³;
with the proviso that at least one amino acid residue among the two amino acid residues at positions P⁹ and P¹⁵ are basic amino acid residues selected from Dab at P⁹, and Dab at P¹⁵.

In some embodiments, the compound of formula (I) is selected from the group consisting of:

(SEQ ID NO: 1)
Gua-Val-Hyp-Ile-Val(3OH)-Tyr-Pen-Asn-Arg-Dab-Thr-
DDab-Lys-Cys-Dab-Arg-Tyr;
(SEQ ID NO: 2)
Gua-Val-Hyp-Ile-Val(3OH)-Tyr-Pen-Ser-Arg-Dab-Thr-
DDab-Lys-Cys-Dab-Arg-Tyr;
(SEQ ID NO: 3)
Gua-Val-Hyp-Ile-Val(3OH)-Tyr-Pen-Asn-Arg-Dab-Ser-
DDab-Lys-Cys-Dab-Arg-Tyr;
(SEQ ID NO: 4)
Gua-Val-Hyp-Ile-Val(3OH)-Tyr-Pen-Asn-Arg-Pro-Ser-
DDab-Dab-Cys-Dab-Dab-Tyr;
(SEQ ID NO: 5)
Gua-Val-Hyp-Ile-Val(3OH)-Tyr-Pen-Asn-Arg-Dab-Thr-
DDab-Dab-Cys-Dab-Dab-Tyr;
(SEQ ID NO: 6)
Gua-Val-Hyp-Ile-Val(3OH)-Tyr-Pen-Asn-Arg-Hyp-Thr-
DDab-Dab-Cys-Dab-Dab-Tyr;
(SEQ ID NO: 7)
Gua-Val-Hyp-Ile-Val(3OH)-Tyr-Pen-Asn-Arg-Hyp-Ser-
DDab-Dab-Cys-Dab-Dab-Tyr;
and
(SEQ ID NO: 8)
Gua-Val-Hyp-Ile-Val(3OH)-Tyr-Pen-Asn-Arg-Dab-Thr-
DDab-Dab-Cys-Dab-Thr-Tyr;

or a salt thereof,
wherein Pen at P⁶ and Cys at P¹³ optionally form a disulfide bridge between P⁶ and P¹³.

In some embodiments, the compound of formula (I) is selected from the group consisting of:

(SEQ ID NO: 4)
Gua-Val-Hyp-Ile-Val(3OH)-Tyr-Pen-Asn-Arg-Pro-Ser-
DDab-Dab-Cys-Dab-Dab-Tyr;
(SEQ ID NO: 5)
Gua-Val-Hyp-Ile-Val(3OH)-Tyr-Pen-Asn-Arg-Dab-Thr-
DDab-Dab-Cys-Dab-Dab-Tyr;
(SEQ ID NO: 6)
Gua-Val-Hyp-Ile-Val(3OH)-Tyr-Pen-Asn-Arg-Hyp-Thr-
DDab-Dab-Cys-Dab-Dab-Tyr;
(SEQ ID NO: 7)
Gua-Val-Hyp-Ile-Val(3OH)-Tyr-Pen-Asn-Arg-Hyp-Ser-
DDab-Dab-Cys-Dab-Dab-Tyr;
and
(SEQ ID NO: 8)
Gua-Val-Hyp-Ile-Val(3OH)-Tyr-Pen-Asn-Arg-Dab-Thr-
DDab-Dab-Cys-Dab-Thr-Tyr;

or a salt thereof;
wherein Pen at P⁶ and Cys at P¹³ optionally form a disulfide bridge between P⁶ and P¹³.

In some embodiments Cys or Pen at P⁶ and Cys or Pen at P¹³ form a disulfide bridge between P⁶ and P¹³. In some embodiments P⁶ is Pen and P¹³ is Cys, which form a disulfide bridge between P⁶ and P¹³.

In some embodiments, the compound of formula (1) is

(SEQ ID NO: 1)
Gua-Val-Hyp-Ile-Val(3OH)-Tyr-Pen-Asn-Arg-Dab-Thr-
DDab-Lys-Cys-Dab-Arg-Tyr,

or a salt thereof, wherein Pen at P⁶ and Cys at P¹³ optionally form a disulfide bridge between P⁶ and P¹³.

In some embodiments, the compound of formula (1) is

(SEQ ID NO: 9)
Gua-Val-Hyp-Ile-Val(3OH)-Tyr-Pen-Asn-Arg-Dab-Thr-
DDab-Lys-Cys-Dab-Arg-Tyr,

or a salt thereof, wherein Pen at P⁶ and Cys at P¹³ form a disulfide bridge between P⁶ and P¹³.

In some embodiments, the compound of formula (1) is

(SEQ ID NO: 2)
Gua-Val-Hyp-Ile-Val(3OH)-Tyr-Pen-Ser-Arg-Dab-Thr-
DDab-Lys-Cys-Dab-Arg-Tyr;

or a salt thereof, wherein Pen at P⁶ and Cys at P¹³ optionally form a disulfide bridge between P⁶ and P¹³.

In some embodiments the compound of formula (1) is

(SEQ ID NO: 10)
Gua-Val-Hyp-Ile-Val(3OH)-Tyr-Pen-Ser-Arg-Dab-Thr-
DDab-Lys-Cys-Dab-Arg-Tyr;

or a salt thereof, wherein Pen at P⁶ and Cys at P¹³ form a disulfide bridge between P⁶ and P¹³.

In some embodiments the compound of formula (1) is

(SEQ ID NO: 3)
Gua-Val-Hyp-Ile-Val(3OH)-Tyr-Pen-Asn-Arg-Dab-Ser-
DDab-Lys-Cys-Dab-Arg-Tyr;

or a salt thereof, wherein Pen at P⁶ and Cys at P¹³ optionally form a disulfide bridge between P⁶ and P¹³.

In some embodiments the compound of formula (1) is

(SEQ ID NO: 11)
Gua-Val-Hyp-Ile-Val(3OH)-Tyr-Pen-Asn-Arg-Dab-Ser-
^{D}Dab-Lys-Cys-Dab-Arg-Tyr;

or a salt thereof, wherein Pen at P⁶ and Cys at P¹³ form a disulfide bridge between P⁶ and P¹³.

In some embodiments the compound is

(SEQ ID NO: 4)
Gua-Val-Hyp-Ile-Val(3OH)-Tyr-Pen-Asn-Arg-Pro-Ser-
^{D}Dab-Dab-Cys-Dab-Dab-Tyr;

or a salt thereof, wherein Pen at P⁶ and Cys at P¹³ optionally form a disulfide bridge between P⁶ and P¹³.

In some embodiments the compound of formula (1) is

(SEQ ID NO: 12)
Gua-Val-Hyp-Ile-Val(3OH)-Tyr-Pen-Asn-Arg-Pro-Ser-
^{D}Dab-Dab-Cys-Dab-Dab-Tyr;

or a salt thereof, wherein Pen at P⁶ and Cys at P¹³ form a disulfide bridge between P⁶ and P¹³.

In some embodiments the compound of formula (I) is

(SEQ ID NO: 5)
Gua-Val-Hyp-Ile-Val(3OH)-Tyr-Pen-Asn-Arg-Dab-Thr-
^{D}Dab-Dab-Cys-Dab-Dab-Tyr;

or a salt thereof, wherein Pen at P⁶ and Cys at P¹³ optionally form a disulfide bridge between P⁶ and P¹³.

In some embodiments the compound of formula (1) is

(SEQ ID NO: 13)
Gua-Val-Hyp-Ile-Val(3OH)-Tyr-Pen-Asn-Arg-Dab-Thr-
^{D}Dab-Dab-Cys-Dab-Dab-Tyr;

or a salt thereof, wherein Pen at P⁶ and Cys at P¹³ form a disulfide bridge between P⁶ and P¹³.

In some embodiments the compound of formula (I) is

(SEQ ID NO: 6)
Gua-Val-Hyp-Ile-Val(3OH)-Tyr-Pen-Asn-Arg-Hyp-Thr-
^{D}Dab-Dab-Cys-Dab-Dab-Tyr;

or a salt thereof, wherein Pen at P⁶ and Cys at P¹³ optionally form a disulfide bridge between P⁶ and P¹³.

In some embodiments the compound of formula (1) is

(SEQ ID NO: 14)
Gua-Val-Hyp-Ile-Val(3OH)-Tyr-Pen-Asn-Arg-Hyp-Thr-
^{D}Dab-Dab-Cys-Dab-Dab-Tyr;

or a salt thereof, wherein Pen at P⁶ and Cys at P¹³ form a disulfide bridge between P⁶ and P¹³.

In some embodiments the compound of formula (1) is

(SEQ ID NO: 7)
Gua-Val-Hyp-Ile-Val(3OH)-Tyr-Pen-Asn-Arg-Hyp-Ser-
^{D}Dab-Dab-Cys-Dab-Dab-Tyr;

or a salt thereof, wherein Pen at P⁶ and Cys at 13 optionally form a disulfide bridge between P⁶ and P¹³.

In some embodiments the compound of formula (1) is

(SEQ ID NO: 15)
Gua-Val-Hyp-Ile-Val(3OH)-Tyr-Pen-Asn-Arg-Hyp-Ser-
^{D}Dab-Dab-Cys-Dab-Dab-Tyr;

or a salt thereof, wherein Pen at P⁶ and Cys at P¹³ form a disulfide bridge between P⁶ and P¹³.

In some embodiments the compound of formula (1) is

(SEQ ID NO: 8)
Gua-Val-Hyp-Ile-Val(3OH)-Tyr-Pen-Asn-Arg-Dab-Thr-
^{D}Dab-Dab-Cys-Dab-Thr-Tyr;

or a salt thereof, wherein Pen at P⁶ and Cys at P¹³ optionally form a disulfide bridge between P⁶ and P¹³.

In some embodiments the compound of formula (I) is

(SEQ ID NO: 16)
Gua-Val-Hyp-Ile-Val(3OH)-Tyr-Pen-Asn-Arg-Dab-Thr-
^{D}Dab-Dab-Cys-Dab-Thr-Tyr;

or a salt thereof, wherein Pen at P⁶ and Cys at P¹³ form a disulfide bridge between P⁶ and P¹³.

In some embodiments one or more atoms are replaced by an atom having an atomic mass number or mass different from the atomic mass number or mass usually found in nature, e.g. compounds enriched in ²H (D), ³H, ¹¹C, ¹⁴C, ¹²⁷I etc. These isotopic analogs and their pharmaceutical salts and formulations are considered useful agents in the therapy and/or diagnostic, for example, but not limited to, where a fine-tuning of in vivo half-life time could lead to an optimized dosage regimen.

The present invention provides compounds with reduced cytotoxicity, increased tolerability, improved bactericidal activity against Enterobacteriaceae including thanatin-resistant clones and/or a reduced propensity to select for resistance.

In some embodiments the compounds of the invention show a lower reduction in binding affinity for LptA protein carrying a Q62L mutation versus the corresponding LptA protein without the mutation, in particular LptA from Escherichia coli and/or Klebsiella pneumoniae.

In some embodiments the compounds of the invention show a lower reduction in efficacy against Gram-negative bacteria resistant to thanatin versus the corresponding non-resistant strain, in particular Escherichia coli and/or Klebsiella pneumoniae.

In some embodiments the compounds of the invention show a lower reduction in efficacy against Gram-negative bacteria harboring an LptA protein carrying a Q62L mutation versus the corresponding strain without the mutation, in particular Escherichia coli and/or Klebsiella pneumoniae.

Hereinafter follows a list of abbreviations, corresponding to generally adopted usual practice, of amino acids or derivatives thereof which, or the residues of which, are suitable for the purposes of the present invention and referred to in this document.

| | | |
|---|---|---|
| Ala | A | L-Alanine |
| Arg | R | L-Arginine |
| Asn | N | L-Asparagine |
| Asp | D | L-Aspartic acid |
| Cys | C | L-Cysteine |
| Gln | Q | L-Glutamine |
| Glu | E | L-Glutamic acid |
| Gly | G | Glycine |
| His | H | L-Histidine |
| Ile | I | L-Isoleucine |
| Leu | L | L-Leucine |
| Lys | K | L-Lysine |
| Met | M | L-Methionine |
| Phe | F | L-Phenylalanine |
| Pro | P | L-Proline |
| Ser | S | L-Serine |
| Thr | T | L-Threonine |
| Trp | W | L-Tryptophan |
| Tyr | Y | L-Tyrosine |
| Val | V | L-Valine |

| | |
|---|---|
| Cit | (S)-2-amino-5-ureidopentanoic acid |
| Cha | (S)-2-amino-3-cyclohexylpropanoic acid |
| Ndab | N-(2-aminoethyl)glycine |
| Nle | (S)-2-aminohexanoic acid |
| Gua-Val | N-amidino-L-valine |
| Pic | (S)-piperidine-2-carboxylic acid |
| Pro((4R)OMe) | (2S,4R)-4-methoxypyrrolidine-2-carboxylic acid |
| Pro(3,4dehydro) | (S)-2,5-dihydro-1H-pyrrole-2-carboxylic acid |
| Dab | (S)-2,4-diaminobutanoic acid |
| Dab(iPr) | (S)-2-amino-4-(isopropylamino)butanoic acid |
| ^{D}Dab(iPr) | (R)-2-amino-4-(isopropylamino)butanoic acid |
| NalloThr | N-((S)-Hydroxyethyl)glycine |
| Orn | (S)-2,5-diaminopentanoic acid |
| Orn(iPr) | (S)-2-amino-5-(isopropylamino)pentanoic acid |
| Pro((4R)NH₂) | (2S,4R)-4-aminopyrrolidine-2-carboxylic acid |
| Hyp | (2S,4R)-4-hydroxypyrrolidine-2-carboxylic acid |
| Val(3OH) | 3-Hydroxy-L-valine |
| Pen | (R)-2-amino-3-mercapto-3-methylbutanoic acid |
| Dap | (5)-2,3-diaminopropanoic acid |

The abbreviation of D-isomers, e.g. ^{D}Dab, refers to the D-enantiomer of the corresponding L-amino acid.

The abbreviation “Gua-” followed by an abbreviation of an amino acid, or amino acid residue, as listed above, corresponds to the N-amidinylated amino acid, or amino acid residue, having the N-terminal amino group replaced by a guanidino (Gua) group. Accordingly, the following abbreviation has the meaning below:
Gua-Val N-amidino-L-Valine

In some embodiments, the pharmaceutical composition is in a form suitable for oral, topical, transdermal, injection, buccal, transmucosal, rectal, pulmonary or inhalation administration. In some embodiments, the pharmaceutical composition is in the form of a tablet, a dragee, a capsule, a solution, a liquid, a gel, a plaster, a cream, an ointment, a syrup, a slurry, a suspension, a spray, a nebulizer, an aerosol, or a suppository.

The compounds of formula (I) may be prepared according to a process which comprises the following steps:
(a) coupling an appropriately functionalized solid support with an appropriately N-protected derivative of that amino acid which in the desired end-product is in position P¹⁶; any functional group which may be present in said N-protected amino acid derivative being likewise appropriately protected;
(b) removing the N-protecting group from the product thus obtained;
   coupling the product thus obtained with an appropriately N-protected derivative of that amino acid which in the desired end-product is in position P¹⁵; any functional group which may be present in said N-protected amino acid derivative being likewise appropriately protected;
(d) effecting steps substantially corresponding to steps (b) and (c) using appropriately N-protected derivatives of amino acids which in the desired end-product are in positions P¹⁴ to P⁶, any functional group(s) which may be present in said N-protected amino acid derivatives being likewise appropriately protected
(e) optionally selectively deprotecting one or several protected functional group(s) present in the molecule and chemically transforming the reactive group(s) thus liberated;
(f) effecting steps substantially corresponding to steps (b) and (c) using appropriately N-protected derivatives of amino acids which in the desired end-product are in positions P⁵ to P², any functional group(s) which may be present in said N-protected amino acid derivatives being likewise appropriately protected; and, optionally, following each coupling, selectively deprotecting one or several protected functional group(s) present in the molecule and chemically transforming the reactive group(s) thus liberated;
(g) further effecting steps substantially corresponding to steps (b) and (c) using an appropriately N-protected derivative of an amino acid, or optionally, an appropriately protected derivative of a hydroxy acid, which in the desired end-product is in position P¹, any functional group(s) which may be present in said N-protected amino acid derivative, or hydroxy acid derivative, being likewise appropriately protected; and, optionally, following the coupling, selectively deprotecting one or several protected functional group(s) present in the molecule and chemically transforming the reactive group(s) thus liberated;
(h) optionally selectively deprotecting one or several protected functional group(s) present in the molecule and chemically transforming the reactive group(s) thus liberated;
(i) optionally, removing the N-protecting group at position P¹;
(j) detaching the product thus obtained from the solid support;
(k) optionally selectively deprotecting one or several protected functional group(s) present in the molecule and chemically transforming the reactive group(s) thus liberated;
(l) removing any protecting groups present on functional groups of any members of the chain of residues and, optionally, any protecting group(s) which may in addition be present in the molecule;
(m) optionally implementing additional chemical transformations of one or more reactive group(s) present in the molecule, e.g. including an oxidative cyclization;
(n) if required, removing any protecting groups present on functional groups of any members of the chain of residues and, optionally, any protecting group(s) which may in addition be present in the molecule; and
(o) optionally converting the product thus obtained into a pharmaceutically acceptable salt; or
   optionally converting a pharmaceutically acceptable or unacceptable salt thus obtained into the corresponding free compound of formula (I); or
   optionally converting a pharmaceutically acceptable or unacceptable salt thus obtained into a different, pharmaceutically acceptable salt.

Enantiomers of the compounds of formula (I) are also part of the present invention. Enantiomers can be prepared by a modification of the above process wherein enantiomers of all chiral starting materials are utilized.

The compounds of formula (I) include all possible tautomeric and rotameric forms.

The compounds of formula (I) may be provided as salts, in particular pharmaceutically acceptable salts. Examples of pharmaceutically acceptable salts of the compounds of the invention are salts of physiologically acceptable mineral acids, such as hydrochloric acid, sulfuric acid and phosphoric acid, or salts of organic acids, such as methane-sulfonic acid, p-toluenesulfonic acid, lactic acid, acetic acid, trifluoroacetic acid, citric acid, succinic acid, fumaric acid, maleic acid and salicylic acid. Further examples of pharmaceutically acceptable salts of the compounds of formula (I) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts.

The compounds of formula (I) and salts thereof may be provided in free form or in the form of a hydrate or a solvate. Hydrates and solvates may be formed during the preparation process.

The process of synthesizing the compounds of formula (I) can advantageously be carried out as parallel array synthesis to yield libraries of peptidomimetics of the invention. Such parallel syntheses allow one to obtain arrays of numerous (normally 12 to 576, typically 96) compounds as described above in moderate to high yields and defined purities, minimizing the formation of dimeric and polymeric by-products.

The functionalized solid support is conveniently derived from polystyrene crosslinked with, preferably 1-5%, divinylbenzene; polystyrene coated with polyethyleneglycol spacers (Tentagel™); and polyacrylamide resins (see also D. Obrecht, J.-M. Villalgordo, “Solid-Supported Combinatorial and Parallel Synthesis of Small-Molecular-Weight Compound Libraries”, Tetrahedron Organic Chemistry Series, Vol. 17, Pergamon, Elsevier Science, 1998).

The solid support is functionalized by means of a linker, i.e. a bifunctional spacer molecule which contains on one end an anchoring group for attachment to the solid support and on the other end a selectively cleavable functional group used for the subsequent chemical transformations and cleavage procedures. For the purposes of the present invention two types of linkers are used:

Type 1 linkers are designed to release the amide group under acidic conditions (H. Rink, Tetrahedron Lett. 1987, 28, 3783-3790). Linkers of this kind form amides of the carboxyl group of the amino acids; examples of resins functionalized by such linker structures include 4-[(((2,4-dimethoxyphenyl) Fmoc-aminomethyl) phenoxyacetamido) amino-methyl] PS resin, 4-[(((2,4-dimethoxyphenyl) Fmoc-aminomethyl) phenoxyacetamido)-aminomethyl]-4-methyl-benzydrylamine PS resin (Rink amide MBHA PS Resin), and 4-[(((2,4-dimethoxy-phenyl) Fmoc-aminomethyl) phenoxyacetamido) aminomethyl] benzhydrylamine PS-resin (Rink amide BHA PS resin), and Fmoc-amino-xanthen-3-yloxy PS resin, Sieber linker resin). Preferably, the support is derived from polystyrene crosslinked with, most preferably 1-5%, divinylbenzene and functionalized by means of the 4-(((2,4-dimethoxy-phenyl) Fmoc-aminomethyl) phenoxyacetamido) linker.

Type 2 linkers are designed to eventually release the carboxyl group under acidic conditions. Linkers of this kind form acid-labile esters with the carboxyl group of the amino acids, usually acid-labile benzyl, benzhydryl and trityl esters; examples of such linker structures include 2-methoxy-4-hydroxymethylphenoxy (Sasrin™ linker), 4-(2,4-dimethoxyphenyl-hydroxy-methyl)-phenoxy (Rink linker), 4-(4-hydroxymethyl-3-methoxyphenoxy) butyric acid (HMPB linker), trityl and 2-chlorotrityl. Preferably, the support is derived from polystyrene crosslinked with, most preferably 1-5%, divinylbenzene and functionalized by means of the 2-chlorotrityl linker.

When carried out as parallel array synthesis the process of the invention can be advantageously carried out as described herein below but it will be immediately apparent to those skilled in the art how these procedures will have to be modified in case it is desired to synthesize one single compound of the invention.

A number of reaction vessels (normally 12 to 576, typically 96) equal to the total number of compounds to be synthesized by the parallel method are loaded with 10 to 1000 mg, preferably 40 mg, of the appropriate functionalized solid support, preferably 1 to 5% cross-linked polystyrene.

The solvent to be used must be capable of swelling the resin and includes, but is not limited to, dichloromethane (DCM), dimethylformamide (DMF), N-methylpyrrolidone (NMP), dioxane, toluene, tetrahydrofuran (THF), ethanol (EtOH), trifluoroethanol (TFE), isopropylalcohol and the like. Solvent mixtures containing as at least one component a polar solvent (e.g. 20% TFE/DCM, 35% THF/NMP) are beneficial for ensuring high reactivity and solvation of the resin-bound peptide chains (G. B. Fields, C. G. Fields, J. Am. Chem. Soc. 1991, 113, 4202-4207).

With the development of various linkers that release the C-terminal carboxylic acid group under mild acidic conditions, not affecting acid-labile groups protecting functional groups in the side chain(s), considerable progresses have been made in the synthesis of protected peptide fragments. The 2-methoxy-4-hydroxybenzylalcohol-derived linker (Sasrin™ linker, Mergler et al., Tetrahedron Lett. 1988, 29, 4005-4008) is cleavable with diluted trifluoroacetic acid (0.5-1% TFA in DCM) and is stable to Fmoc deprotection conditions during the peptide synthesis, Boc/tBu-based additional protecting groups being compatible with this protection scheme. Other linkers which are suitable for the process of the invention include the super acid labile 4-(2,4-dimethoxyphenyl-hydroxymethyl)-phenoxy linker (Rink linker, H. Rink, Tetrahedron Lett. 1987, 28, 3787-3790), where the removal of the peptide requires 10% acetic acid in DCM or 0.2% trifluoroacetic acid in DCM; the 4-(4-hydroxymethyl-3-methoxyphenoxy) butyric acid-derived linker (HMPB-linker, Flörsheimer & Riniker, 1991, Peptides 1990:

Proceedings of the Twenty-First European Peptide Symposium, 131) which is also cleaved with 1% TFA/DCM in order to yield a peptide fragment containing all acid labile side-chain protective groups; and, in addition, the 2-chlorotritylchloride linker (Barlos et al., Tetrahedron Lett. 1989, 30, 3943-3946), which allows the peptide detachment using a mixture of glacial acetic acid/trifluoroethanol/DCM (1:2:7) for 30 min.

Suitable protecting groups for amino acids and, respectively, for their residues are, for example,
for the amino group (as is present e.g. also in the side-chain of lysine)

| | |
|---|---|
| Cbz | benzyloxycarbonyl |
| Boc | tert-butyloxycarbonyl |
| Fmoc | 9-fluorenylmethoxycarbonyl |
| Alloc | allyloxycarbonyl |
| Teoc | trimethylsilylethoxycarbonyl |
| Tcc | trichloroethoxycarbonyl |
| Nps | o-nitrophenylsulfonyl |
| Trt | triphenylmethyl or trityl |
| ivDe | 1-(4,4-dimethyl-2,6-dioxocyclohex-1-ylidene)-3-methylbutyl; |

for the carboxyl group (as is present e.g. also in the side-chain of aspartic and glutamic acid) by conversion into esters with the alcohol components

| | |
|---|---|
| tBu | tert-butyl |
| Bn | benzyl |
| Me | methyl |
| Ph | phenyl |
| Pac | phenacyl |
| | allyl |
| Tse | trimethylsilylethyl |
| Tce | trichloroethyl |
| Dmab | 4-N-(1-[dimethyl-2,6-dioxocyclohexylidene]-3-methylbutyl)- |
| | amino benzyl; |
| 2-PhiPr | 2-phenyl-isopropyl; |

for the guanidino group (as is present e.g. in the side-chain of arginine)

| | |
|---|---|
| Pmc | 2,2,5,7,8-pentamethylchroman-6-sulfonyl |
| Ts | tosyl (i.e. p-toluenesulfonyl) |
| Cbz | benzyloxycarbonyl |
| Pbf | pentamethyldihydrobenzofuran-5-sulfonyl; |

and for the hydroxy group (as is present e.g. in the side-chain of threonine and serine)

| | |
|---|---|
| tBu | tert-butyl |
| Bn | benzyl |
| Trt | trityl |
| Alloc | allyloxycarbonyl. |

The 9-fluorenylmethoxycarbonyl-(Fmoc)-protected amino acid derivatives are preferably used as the building blocks for the construction of the peptidomimetics of the invention. For the deprotection, i.e. cleaving off the Fmoc group, 20% piperidine in DMF or 2% DBU in DMF can be used.

The quantity of the reactant, i.e. of the amino acid derivative, is usually 1 to 20 equivalents (eq) based on the milliequivalents per gram (meq/g) loading of the functionalized solid support (typically 0.1 to 2.85 meq/g for polystyrene resins) originally weighed into the reaction tube. Additional equivalents of reactants can be used, if required, to drive the reaction to completion in a reasonable time. The preferred workstations (without, however, being limited thereto) are Protein Technologies' Symphony X and MultiSynTech's-Syro synthesizer, the latter additionally equipped with a transfer unit and a reservoir box during the process of detachment of the fully protected linear peptide from the solid support. All synthesizers are able to provide a controlled environment, for example, reactions can be accomplished at temperatures different from room temperature as well as under inert gas atmosphere, if desired.

Amide bond formation requires the activation of the α-carboxyl group for the acylation step. When this activation is being carried out by means of the commonly used carbodiimides such as dicyclohexylcarbodiimide (DCC, Sheehan & Hess, J. Am. Chem. Soc. 1955, 77, 1067-1068) or diisopropylcarbodiimide (DIC, Sarantakis et al Biochem. Biophys. Res. Commun. 1976, 73, 336-342), the resulting dicyclohexylurea and, respectively, diisopropylurea is insoluble and, respectively, soluble in the solvents generally used. In a variation of the carbodiimide method, 1-hydroxy benzotriazole (HOBt, König & Geiger, Chem. Ber. 1970, 103, 788-798) or HOAt or ethyl cyano (hydroxyimino) acetate (Oxyma, (R. Subirós-Funosas, et al, Chem. Eur. J. 2009, 15, 9394-9403)) is included as an additive to the coupling mixture. HOBt, HOAt and Oxyma prevent dehydration, suppresses racemization of the activated amino acids and acts as a catalyst to improve the sluggish coupling reactions. Certain phosphonium reagents have been used as direct coupling reagents, such as benzotriazol-1-yl-oxy-tris-(dimethyl-amino)-phosphonium hexafluorophosphate (BOP, Castro et al., Tetrahedron Lett. 1975, 14, 1219-1222; Synthesis 1976, 751-752), or benzotriazol-1-yl-oxy-tris-pyrrolidino-phosphonium hexaflurophoshate (Py-BOP, Coste et al., Tetrahedron Lett. 1990, 31, 205-208), or 2-(1H-benzotriazol-1-yl-) 1,1,3,3-tetramethyluronium tetrafluoroborate (TBTU), or hexafluorophosphate (HBTU, Knorr et al., Tetrahedron Lett. 1989, 30, 1927-1930); these phosphonium reagents are also suitable for in situ formation of HOBt esters with the protected amino acid derivatives. Diphenoxyphosphoryl azide (DPPA) or O-(7-aza-benzotriazol-1-yl)-N,N,N′,N′-tetramethyluronium tetrafluoro borate (TATU) or O-(7-aza-benzotriazol-1-yl)-N,N,N′,N′-tetramethyluronium hexa fluorophosphate (HATU)/7-aza-1-hydroxybenzotriazole (HOAt, Carpino et al., Tetrahedron Lett. 1994, 35, 2279-2281) or -(6-Chloro-1H-benzotriazol-1-yl-)-N,N,N′,N′-1,1,3,3-tetramethyl uronium tetrafluoroborate (TCTU), or hexafluoro phosphate (HCTU, Marder, Shivo and Albericio: HCTU and TCTU: New Coupling Reagents: Development and Industrial Applications, Poster Presentation, Gordon Conference February 2002) can be used as coupling reagents as well as 1,1,3,3-bis(tetramethylene) chlorouronium hexafluorophosphate (PyCIU) especially for coupling of N-methylated amino acids (J. Coste, E. Frérot, P. Jouin, B. Castro, Tetrahedron Lett. 1991, 32, 1967) or pentafluorophenyl diphenyl-phosphinate (S. Chen, J. Xu, Tetrahedron Lett. 1991, 32, 6711). More recently, new coupling reagents based on Oxyma have been introduced e.g ([(1-(cyano-2-ethoxy-2-oxoethyl-ideneaminooxy) dimethylaminomorpholino)] uronium hexafluorophosphate (COMU, A. El-Faham, et al. Chem. Eur. J 2009, 15, 9404-9416))

Due to the fact that near-quantitative coupling reactions are essential, it is desirable to have experimental evidence for completion of the reactions. The ninhydrin test (Kaiser et al., Anal. Biochemistry 1970, 34, 595) and the 2,4,6-trinitrobenzene sulfonic (TNBS) test (Hancook W. S. et al, Anal. Biochem 1976, 71, 260), where a positive colorimetric response to an aliquot of resin-bound peptide or peptide indicates qualitatively the presence of the primary amine, can easily and quickly be performed after each coupling step. For the secondary amine detection e.g for proline derivatives, the chloranil test (Vojkovsky T., Pept. Res. 1995, 68, 236) can be used. Fmoc chemistry allows the spectrophotometric detection of the Fmoc chromophore when it is released with the base (Meienhofer et al., Int. J. Peptide Protein Res. 1979, 13, 35-42).

The resin-bound intermediate within each reaction vessel is washed free of excess of retained reagents, of solvents, and of by-products by repetitive exposure to pure solvent(s).

Washing procedures are repeated up to about 30 times (preferably about 5 times), monitoring the efficiency of reagent, solvent, and by-product removal by methods such as TLC, GC, LC-MS or inspection of the washings.

The above described procedure of reacting the resin-bound compound with reagents within the reaction wells followed by removal of excess reagents, by-products, and solvents is repeated with each successive transformation until the final resin-bound fully protected linear peptide has been obtained.

Finally, after the on-support synthesis including elongation and modification e.g N-terminal functionalization or cyclization, the concomitant detachment and full deprotection of the peptide derivative can be performed with 95% TFA, 2.5% H₂O, 2.5% TIS, or 82.5% TFA, 5% anisole, 5% thioanisole, 5% H₂O and 2.5% TIS or another combination of scavengers for effecting the cleavage of the protected peptide and removal of protecting groups. The deprotection reaction time is commonly 30 minutes to 12 hours, preferably about 2.5 hours. The deprotected linear or cyclic peptide can be precipitated and washed using cold Et₂O or isopropyl ether (IPE).

For some compounds of the present invention according to general formula (I) additional synthetic steps are required. These transformations can be applied either on a fully protected or partially deprotected linear or cyclic peptide, attached to or already released from the solid support or on the final deprotected molecule.

A widely known linkage is the disulfide bridge formed by e.g. cysteines, homo-cysteines or penicillamine (Pen). To form the disulfide bridge from sulfur amino acids such as Cys or Pen, oxidation reactions are performed according to general reaction conditions procedures known to person skilled in the art. Various oxidants can be used for rapid formation of disulfide bridges to desire cyclic peptides. These include potassium ferricyanide (Rüegg et al, Hoppe-Seyler's Zeitschrift für physiologische Chemie. 1975, 356, 1527-1534, iodine (Zhang et al, Internat. J. Peptide Res. Ther. 2008, 14, 301-305), DMSO (Beekman et al, J. Pep. Res, 2009, 50, 357) and N-chlorosuccinimide (NCS) (Mc Curdy, Pept Res. 1989, 2, 147-152).

Typically, the peptide is dissolved in ammonium acetate buffer 1M at pH 6 containing DMSO and stirred for 24 to 48 h to obtain cyclic peptide. The peptide can also be dissolved in a water solution containing 10% of acetic acid, in presence of I₂ and KI at room temperature. The oxidation reaction is performed before purification of the peptides according to procedure D (see below).

Depending on its purity, the final product as obtained following the procedures above can be used directly for biological assays, or if desired can be further purified, for example by preparative HPLC.

It is thereafter possible, if desired, to convert the fully deprotected product thus obtained into a pharmaceutically acceptable salt or a non-pharmaceutically acceptable salt, or to convert a salt thus obtained into the corresponding free compound or into a different salt. Any of these operations can be carried out by methods well known in the art.

Peptides are usually obtained as their TFA salt due to the nature of peptide cleavage reaction conditions and reversed-phase purification techniques. Ion-exchange chromatography is a standard approach to convert peptide TFA salts to pharmaceutically acceptable salts, such as acetate and HCl salt. Standard procedures, known to person skilled in the art, can be followed to prepare desired pharmaceutically acceptable salts (Roux at al, J. Pep. Sci., 2008, 14, 354-359).

In general, the building blocks for the peptide derivatives of the present invention can be synthesized according to the literature methods known to a person skilled in the art or can be obtained from commercial sources. All other corresponding amino acids have been described either as unprotected or as Boc- or Fmoc-protected racemates, (D)- or (L)-isomers. It will be appreciated that unprotected amino acid building blocks can be easily transformed into the corresponding Fmoc-protected amino acid building blocks required for the present invention by standard protecting group manipulations. Reviews describing general methods for the synthesis of α-amino acids include: R. Duthaler, Tetrahedron (Report) 1994, 349, 1540-1650; R. M. Williams, “Synthesis of optically active α-amino acids”, Tetrahedron Organic Chemistry Series, Vol. 7, J. E. Baldwin, P. D. Magnus (Eds.), Pergamon Press., Oxford 1989. An especially useful method for the synthesis of optically active α-amino acids relevant for this invention includes kinetic resolution using hydrolytic enzymes (M. A. Verhovskaya, I. A. Yamskov, Russian Chem. Rev. 1991, 60, 1163-1179; R. M. Williams, “Synthesis of optically active α-amino acids”, Tetrahedron Organic Chemistry Series, Vol. 7, J. E. Baldwin, P. D. Magnus (Eds.), Pergamon Press., Oxford 1989, Chapter 7, p. 257-279). Kinetic resolution using hydrolytic enzymes involves hydrolysis of amides and nitriles by aminopeptidases or nitrilases, cleavage of N-acyl groups by acylases, and ester hydrolysis by lipases or proteases. It is well documented that certain enzymes will lead specifically to pure (L)-enantiomers whereas others yield the corresponding (D)-enantiomers (e.g.: R. Duthaler, Tetrahedron Report 1994, 349, 1540-1650; R. M. Williams, “Synthesis of optically active α-amino acids”, Tetrahedron Organic Chemistry Series, Vol. 7, J. E. Baldwin, P. D. Magnus (Eds.), Pergamon Press., Oxford 1989).

The peptidomimetics of the invention can be used in a wide range of applications in order to inhibit the growth of or to kill microorganisms leading to the desired therapeutic effect in man or, due to their similar etiology, in other mammals. In particular they can be used to inhibit the growth of or to kill Gram-negative bacteria, in particular Enterobacteriaceae, and even more particular Klebsiella pneumoniae and/or Escherichia coli.

“Treatment” or “treating” as used herein in the context of treating a bacterial infection, pertains generally to treatment and therapy, whether of a human or an animal (e.g. in veterinary applications), in which a desired therapeutic effect is achieved, for example, the inhibition of the progress of the infection, and includes a reduction in the rate of progress, a halt in the rate of progress, alleviation of symptoms of the infection, amelioration of the infection, and cure of the infection. “Prevent”, “preventing” or “prevention” as used herein comprises the prevention of at least one symptom associated with or caused by the bacterial infection being prevented.

The peptidomimetics of the invention can be used for example as disinfectants or as preservatives for materials such as foodstuffs, cosmetics, medicaments and other nutrient-containing materials.

The peptidomimetics of the invention can also be used to treat or prevent diseases related to microbial infection in plants and animals.

For use as disinfectants or preservatives the peptidomimetics can be added to the desired material singly, as mixtures of several peptidomimetics or in combination with other antimicrobial agents.

The peptidomimetics of the invention can be used to treat or prevent infections or diseases related to such infections, in particular infections or diseases related to such infections caused by Gram-negative bacteria, in particular Enterobacteriaceae, and even more particular Klebsiella pneumoniae and/or Escherichia coli. The peptidomimetics of the invention can be used to treat or prevent nosocomial infections caused by Gram-negative bacteria related to diseases such as ventilator-associated pneumonia (VAP), hospital-acquired pneumonia (HAP), healthcare-associated pneumonia (HCAP); catheter-related and non-catheter-related infections such as urinary tract infections (UTIs) or bloodstream infections (BSIs); infection-induced sepsis; infections related to respiratory diseases such as cystic fibrosis, emphysema, asthma or pneumonia; infections related to skin or soft tissue diseases such as surgical wounds, traumatic wounds or burn; infections related to gastrointestinal diseases such as epidemic diarrhea, necrotizing enterocolitis, typhlitis, gastroenteritis or pancreatitis; infections related to eye diseases such as keratitis and endophthalmitis; infections related to ear diseases such as otitis; infections related to CNS diseases such as brain abscess and meningitis or encephalitis; infections related to bone diseases such as osteochondritis and osteomyelitis; infections related to cardiovascular diseases such as endocartitis and pericarditis; or infections related to genitourinary diseases such as epididymitis, prostatitis and urethritis. They can be administered singly, as mixtures of several peptidomimetics, in combination with other antimicrobial or antibiotic agents, or anti cancer agents, or antiviral (e.g. anti-HIV) agents, or in combination with other pharmaceutically active agents. The peptidomimetics can be administered per se or as pharmaceutical compositions.

The term “infections related to diseases” can be used interchangeably with and/or embraces the meaning of the terms “infections associated with diseases” and “infections accompanying diseases”. The same applies for a specific infection or infections related to a specific disease or diseases, e.g., infections related to CNS diseases such as brain abscess and meningitis or encephalitis.

The term “diseases related to such infections” can be used interchangeably with and/or embraces the meaning of the terms “diseases associated with such infections” and “diseases accompanied by such infections”. The same applies for a specific disease or diseases related to a specific infection or infections, e.g., nosocomial infections caused by Gram-negative bacteria related to diseases such as ventilator-associated pneumonia (VAP), hospital-acquired pneumonia (HAP), etc.

It is understood that the peptidomimetics of the invention exert their antibacterial activity in the same way as thanatin, namely by disrupting the lipopolysaccharide transport protein bridge in Gram-negative bacteria. In particular, thanatin acts as a competitive inhibitor of the protein-protein interactions mediating the lipopolysaccharide transport bridge assembly, thereby inhibiting lipopolysaccharide transport across the periplasm (Schuster et al., Sci. Adv. 9, eadg3683 (2023)).

The peptidomimetics of the invention may be administered per se or may be applied as an appropriate formulation together with carriers, diluents or excipients well known in the art.

Pharmaceutical compositions comprising peptidomimetics of the invention may be manufactured by means of conventional mixing, dissolving, granulating, coated tablet-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active peptidomimetics into preparations which can be used pharmaceutically. Proper formulation depends upon the method of administration chosen.

For topical administration the peptidomimetics of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injections, the peptidomimetics of the invention may be formulated in adequate solutions, preferably in physiologically compatible buffers such as Hink's solution, Ringer's solution, or physiological saline buffer. The solutions may contain pharmaceutical excipients such as suspending, stabilizing and/or dispersing agents. Alternatively, the peptidomimetics of the invention may be in powder form for combination with a suitable vehicle, e.g., sterile pyrogen-free water, before use.

For transmucosal administration, penetrants appropriate to the barrier to be permeated are used in the formulation as known in the art.

For oral administration, the compounds can be readily formulated by combining the active peptidomimetics of the invention with pharmaceutically acceptable carriers well known in the art. Such carriers enable the peptidomimetics of the invention to be formulated as tablets, pills, dragees, capsules, liquids, gels, syrups, slurries, suspensions etc., for oral ingestion by a patient to be treated. For oral formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl cellulose, sodium carboxymethyl-cellulose, and/or polyvinylpyrrolidone (PVP); granulating agents; and binding agents. If desired, disintegrating agents may be added, such as cross-linked polyvinylpyrrolidones, agar, or alginic acid or a salt thereof, such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. In addition, flavoring agents, preservatives, coloring agents and the like may be added.

For buccal administration, the composition may take the form of tablets, lozenges, etc. formulated as usual.

For administration by inhalation, the peptidomimetics of the invention are conveniently delivered in form of an aerosol spray from pressurized packs or a nebulizer, with the use of a suitable propellant, e.g. dichlorodifluoromethane, trichlorofluromethane, carbon dioxide or another suitable gas. In the case of a pressurized aerosol the dose unit may be determined by providing a valve to deliver a metered amount. Capsules and cartridges of e.g. gelatin for use in an inhaler or insufflator may be formulated containing a powder mix of the peptidomimetics of the invention and a suitable powder base such as lactose or starch.

The compounds may also be formulated in rectal or vaginal compositions such as suppositories together with appropriate suppository bases such as cocoa butter or other glycerides.

In addition to the formulations described above, the peptidomimetics of the invention may also be formulated as depot preparations. Such long-acting formulations may be administered by implantation (e.g. subcutaneously or intramuscularly) or by intramuscular injection. For the manufacture of such depot preparations the peptidomimetics of the invention may be formulated with suitable polymeric or hydrophobic materials (e.g. as an emulsion in an acceptable oil) or ion exchange resins, or as sparingly soluble salts.

In addition, other pharmaceutical delivery systems may be employed such as liposomes and emulsions well known in the art. Certain organic solvents such as dimethylsulfoxide may also be employed. Additionally, the peptidomimetics of the invention may be delivered using a sustained-release system, such as semipermeable matrices of solid polymers containing the therapeutic agent (e.g. for coated stents). Various sustained-release materials have been established and are well known by those skilled in the art. Sustained-release capsules may, depending on their chemical nature, release the compounds for a few weeks up to over 100 days. Depending on the chemical nature and the biological stability of the therapeutic agent, additional strategies for protein stabilization may be employed.

As the peptidomimetics of the invention may contain charged residues, they may be included in any of the above-described formulations as such or as pharmaceutically acceptable salts. Pharmaceutically acceptable salts tend to be more soluble in aqueous and other protic solvents than are the corresponding free base forms.

The peptidomimetics of the invention, or compositions thereof, will generally be used in an amount effective to achieve the intended purpose. It is to be understood that the amount used will depend on a particular application.

For example, for use as a disinfectant or preservative, an antimicrobially effective amount of a peptidomimetic of the invention, or a composition thereof, is applied or added to the material to be disinfected or preserved. By antimicrobially effective amount is meant an amount of a peptidomimetic of the invention, or a composition thereof, that inhibits the growth of, or is lethal to, a target microbe population. While the antimicrobially effective amount will depend on a particular application, for use as disinfectants or preservatives the peptidomimetics of the invention, or compositions thereof, are usually added or applied to the material to be disinfected or preserved in relatively low amounts. Typically, the peptidomimetics of the invention comprise less than about 5% by weight of a disinfectant solution or material to be preserved, preferably less than 1% by weight and more preferably less than 0.1% by weight. An ordinary skilled expert will be able to determine antimicrobially effective amounts of particular peptidomimetics of the invention for particular applications without undue experimentation using, for example, the results of the in vitro assays provided in the examples.

For use to treat or prevent microbial infections or diseases related to such infections, the peptidomimetics of the invention, or compositions thereof, are administered or applied in a therapeutically effective amount. By therapeutically effective amount is meant an amount effective in ameliorating the symptoms of, or in ameliorating, treating or preventing microbial infections or diseases related thereto. Determination of a therapeutically effective amount is well within the capacities of those skilled in the art, especially in view of the detailed disclosure provided herein.

As in the case of disinfectants and preservatives, for topical administration to treat or prevent bacterial infections and/or viral infections a therapeutically effective dose can be determined using, for example, the results of the in vitro assays provided in the examples. The treatment may be applied while the infection is visible, or even when it is not visible. An ordinary skilled expert will be able to determine therapeutically effective amounts to treat topical infections without undue experimentation.

For systemic administration, a therapeutically effective dose can be estimated initially from in vitro assays. For example, a dose can be formulated in animal models to achieve a circulating peptidomimetic concentration range that includes the IC₅₀ as determined in the cell culture (i.e. the concentration of a test compound that is lethal to 50% of a cell culture). Such information can be used to more accurately determine useful doses in humans.

Initial dosages can also be determined from in vivo data, e.g. animal models, using techniques that are well known in the art. One having ordinary skill in the art could readily optimize administration to humans based on animal data.

Dosage amounts for applications as anti-infective agents may be adjusted individually to provide plasma levels of the peptidomimetics of the invention which are sufficient to maintain the therapeutic effect. Therapeutically effective serum levels may be achieved by administering multiple doses each day.

In cases of local administration or selective uptake, the effective local concentration of the peptidomimetics of the invention may not be related to plasma concentration. One having the ordinary skill in the art will be able to optimize therapeutically effective local dosages without undue experimentation.

The amount of peptidomimetics administered will, of course, be dependent on the subject being treated, on the subject's weight, the severity of the affliction, the manner of administration and the judgement of the prescribing physician. For example, in the case of oral administration a daily dosage of about 1 to 1000 mg per person of a compound of the invention may be appropriate, although the above lower and/or upper limit can also be exceeded when necessary.

The antimicrobial therapy may be repeated intermittently while infections are detectable or even when they are not detectable. The therapy may be provided alone or in combination with other drugs, such as for example anti-HIV agents or anti-cancer agents, or other antimicrobial agents.

Normally, a therapeutically effective dose of the peptidomimetics described herein will provide therapeutic benefit without causing substantial toxicity.

Toxicity of the peptidomimetics of the invention can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in humans. The dosage of the peptidomimetics of the invention lies preferably within a range of circulating concentrations that include the effective dose with little or no toxicity. The dosage may vary within the range depending upon the dosage form employed and the route of administration utilized. The exact formulation, route of administration and dose can be chosen by the individual physician in view of the patient's condition (see, e.g. Fingl et al. 1975, In: The Pharmacological Basis of Therapeutics, Ch. 1, p. 1).

The subject to be treated by the compounds the present invention may be a human or animal, e.g. a mammal, and is preferably a human.

Reference to microbial infections, antimicrobial therapy, antimicrobial activity, antimicrobial agents preferably refer in each case to bacterial infections, antibacterial therapy, antibacterial activity and antibacterial agents, wherein the bacteria are preferably gram-negative bacteria.

The schemes and processes described herein are not intended to present an exhaustive list of methods for preparing the compounds of formula (I); rather, additional techniques of which the skilled chemist is aware of may be also used for the compound synthesis.

All aspects and embodiments of the invention described herein may be combined in any combination where possible.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

### BRIEF DESCRIPTION OF THE FIGURES

FIG. 1

FIG. 1A shows efficacy of Example 4 and levofloxacin in the K. pneumoniae ATCC 43816 lung infection model: bar chart. FIG. 1B shows efficacy of Example 4 and levofloxacin in the K. pneumoniae ATCC 43816 lung infection model: change in bacterial count. Data represent the change in bacterial counts in lung tissue following test article treatment relative to the initial 2 h CFU/lung at the time of dosing (baseline counts).

Test animals were rendered neutropenic with IP cyclophosphamide administration, 150 mg/kg on Day-4 then 100 mg/kg on Day-1 prior to infection on Day 0. On Day 0, animals were intranasally inoculated with 1.02×10⁵ CFU/mouse (0.02 mL/animal) of the K. pneumoniae ATCC 43816 strain. The vehicle and Example 4 at 0.25, 0.5, 1.25, 2.5, 5, 6.25, 7.5, 8.75, 10, 11.25, 12.5 was intravenously (IV) slow bolus (30 s) administered twice with a 12 h interval (BID q12 h) at 2 and 14 h after infection. The control reference agent, levofloxacin at 40 mg/kg, was subcutaneously (SC) administered BID q12 h at 2 and 14 h after infection. Animals were sacrificed at 2 or 26 h post-infection, and the lung tissues were harvested and weighed from each of the test animals. The bacterial counts (CFU/Lung) of lung tissue homogenates were measured.

(*): Indicates a significant difference (p<0.05) between the vehicle control and treatment group was determined by one-way ANOVA followed by Dunnett's test.

Error bars are standard error of the mean (SEM) values.

FIG. 2

FIG. 2 shows Example 4 efficacy in the K. pneumoniae ATCC 43816 lung infection model: data analysis with the Hill equation. Data are the bacterial counts in the lung tissues at 24 h after the intravenous (IV) treatments of Example 4 at 0.25, 0.5, 1.25, 2.5, 5, 6.25, 7.5, 8.75, 10, 11.25, 12.5, and 15 mg/kg twice with 12 h intervals (BID q12 h) starting at 2 h after infection for one day. The tested doses (mg/kg) are plotted on the X axis and the response (CFU/Lung) is plotted on the Y axis, both using the logarithm (log 10) scale to generate a sigmoidal plot. The bacteriostatic (baseline level), 1-log 10 and 2-log 10 reduction in bacterial counts relative to the initial 2 h counts was calculated with non-linear regression using the variable slope Hill equation [Y=Bottom+(Top−Bottom)/(1+10 (Log IC50−X)*HillSlope))] by GraphPad® Prism 8.0. Top and Bottom are plateaus in Y axis values with the following best-fit estimates: Bottom (1.00), Top (8.790), Log_{IC50} (0.2182), HillSlope (−1.220); IC50 (1.653), and Span (Top−Bottom, 7.79).

FIG. 3

FIG. 3A shows efficacy of Example 8 and levofloxacin in the K. pneumoniae ATCC 43816 lung infection model: bar chart. FIG. 3B shows efficacy of Example 8 and levofloxacin in the K. pneumoniae ATCC 43816 lung infection model: change in bacterial count. Data represent the change in bacterial counts in lung tissue following test article treatment relative to the initial 2 h CFU/lung at the time of dosing (baseline counts).

Test animals were rendered neutropenic with IP cyclophosphamide administration, 150 mg/kg on Day −4 then 100 mg/kg on Day −1 prior to infection on Day 0. On Day 0, animals were intranasally inoculated with 1.02×10⁵ CFU/mouse (0.02 mL/animal) of the K. pneumoniae ATCC 43816 strain. The vehicle and Example 8 at 0.25, 0.5, 1.25, 2.5, 5, 6.25, 7.5, 8.75, 10, 12.5, 15, and 17.5 mg/kg was intravenously (IV) slow bolus (30 s) administered twice with a 12 h interval (BID q12 h) at 2 and 14 h after infection. The control reference agent, levofloxacin at 40 mg/kg, was subcutaneously (SC) administered BID q12 h at 2 and 14 h after infection. Animals were sacrificed at 2 or 26 h post-infection, and the lung tissues were harvested and weighed from each of the test animals. The bacterial counts (CFU/Lung) of lung tissue homogenates were measured.

(*): Indicates a significant difference (p<0.05) between the vehicle control and treatment group was determined by one-way ANOVA followed by Dunnett's test.

Error bars are SEM values.

FIG. 4

FIG. 4 shows Example 8 efficacy in the K. pneumoniae ATCC 43816 lung infection model: data analysis with the Hill equation. Data are the bacterial counts in the lung tissues at 24 h after the intravenous (IV) treatments of Example 8 at 0.25, 0.5, 1.25, 2.5, 5, 6.25, 7.5, 8.75, 10, 12.5, 15, and 17.5 mg/kg twice with 12 h intervals (BID q12 h) starting at 2 h after infection for one day. The tested doses (mg/kg) are plotted on the X axis and the response (CFU/Lung) is plotted on the Y axis, both using the logarithm (log 10) scale to generate a sigmoidal plot. The bacteriostatic (baseline level), 1-log 10 and 2-log 10 reduction in bacterial counts relative to the initial 2 h counts was calculated with non-linear regression using the variable slope Hill equation [Y=Bottom+(Top−Bottom)/(1+10 (Log_{IC50}−x)*HillSlope))] by GraphPad® Prism 8.0. Top and Bottom are plateaus in Y axis values with the following best-fit estimates: Bottom (1.316), Top (9.679), Log_{IC50} (0.02436), HillSlope (−1.571); IC50 (1.058), and Span (Top−Bottom, 8.363).

FIG. 5

FIG. 5A shows efficacy of Example 8 and colistin (CST) in the K. pneumoniae NCTC 13443 lung infection model: bar chart. FIG. 5B shows efficacy of Example 8 and colistin (CST) in the K. pneumoniae NCTC 13443 lung infection model: change in bacterial count. Data represents the change in bacterial counts in lung tissue following test article treatment relative to the initial 2 h CFU/lung at the time of dosing (baseline counts).

Prior to infection, test animals were rendered neutropenic with IP cyclophosphamide administration, 150 mg/kg on Day −4 and 100 mg/kg on Day −1. On Day 0, animals were intranasally inoculated with 5.6×10⁵ CFU/mouse (0.02 mL/animal) of the K. pneumoniae NCTC 13443 strain. The vehicle and Example 8 at 0.25, 0.5, 1.25, 2.5, 5, 6.25, 7.5, 8.75, 10, 12.5, 15, and 17.5 mg/kg, were intravenously (IV) slow bolus (30 s) administered twice with a 12 h interval (BID q12 h) at 2 and 14 h after infection. The control reference agent, colistin at 40 mg/kg, was subcutaneously (SC) administered BID q12 h at 2 and 14 h after infection. Animals were sacrificed at 2 or 26 h post-infection, and the lung tissues were harvested and weighed from each of the test animals. The bacterial counts (CFU/Lung) of lung tissue homogenates were measured.

(*): Indicates a significant difference (p<0.05) between the vehicle control and treatment group was determined by one-way ANOVA followed by Dunnett's test. Percentage (%) in parenthesis indicates the percentage of animals with bacterial counts below the limit of detection (LOD).

Error bars are SEM values.

FIG. 6

FIG. 6 shows Example 8 efficacy in the K. pneumoniae NCTC 13443 lung infection model: data analysis with the Hill equation. Data are the bacterial counts in the lung tissues at 24 h after the intravenous (IV) treatments of Example 8 at 0.25, 0.5, 1.25, 2.5, 5, 6.25, 7.5, 8.75, 10, 12.5, 15, and 17.5 mg/kg twice with 12 h intervals (BID q12 h) starting at 2 h after infection for one day. The tested doses (mg/kg) are plotted on the X axis and the response (CFU/Lung) is plotted on the Y axis, both using the logarithm (log 10) scale to generate a sigmoidal plot. The bacteriostatic (baseline level), 1-log 10 and 2-log 10 reduction in bacterial counts relative to the initial 2 h counts was calculated with non-linear regression using the variable slope Hill equation [Y=Bottom+(Top−Bottom)/(1+10 (Log IC50−X)*HillSlope))] by GraphPad® Prism 8.0. Top and Bottom are plateaus in Y axis values with the following best-fit estimates: Bottom (2.559), Top (7.459), Log_{IC50} (0.2395), HillSlope (−3.448); IC50 (1.736), and Span (Top−Bottom, 4.900). The following Examples illustrate the present invention but are not to be construed as limiting its scope in any way.

Abbreviations:

| | |
|---|---|
| Ac | Acetyl; |
| BSA | Bovine serum albumin; |
| Boc | tert-Butyloxycarbonyl; |
| DCHA | Dicyclohexylamine; |
| DCM | Dichloromethane; |
| DEAD | Diethyl azodicarboxylate; |
| DIC | Diisopropylcarbodiimid; |
| DIPEA | Diisopropylethylamine; |
| DMF | Dimethylformamide; |
| DMEM | Dulbecco's Modified Eagle's Medium; |
| DODT | 3,6-dioxa-1,8-octanedithiol; |
| FCS | Fetal Calf Serum; |
| Fmoc | Fluorenylmethyloxycarbonyl; |
| HATU | O-(7-Aza-benzotriazole-1-yl)-N,N,N′,N′-tetramethyluronoium |
| | hexafluorophosphate; |
| HBSS | Hank's Buffered Salt Solution; |
| HBTU | O-(Benzotriazol-1-yl)-N,N,N′,N′-tetramethyluronium |
| | hexafluorophosphate; |
| HCTU | O-(6-Chlorobenzotriazol-1-yl)-N,N,N′,N′-tetramethyluronium |
| | hexafluorophosphate; |
| Hepes | 4-(2-hydroxyethyl)-1-piperazineethanesulfonic acid; |
| HFIP | Hexafluoroisopropanol |
| HOAt | 1-Hydroxy-7-azabenzotriazole; |
| IMDM | Iscove's Modified Dulbecco's Media; |
| IPE | Isopropylether; |
| iPrOH | Isopropanol |
| NMP | N-Methyl-2-pyrrolidone; |
| NMM | N-Methylmorpholine; |
| Oxyma | Ethylcyanohydroxyiminoacetate; |
| PyBop ® | (Benzotriazol-1-yloxy)tripyrrolidinophosphonium |
| | hexafluorophosphate; |
| TIS | Triisopropylsilane; |
| TPP | Triphenylphosphine; |
| RPMI | Roswell Park Memorial Institute medium; |
| rt | Room temperature. |

### EXAMPLES

### 1. Peptide Synthesis

### 1.1 General Synthetic Procedures

A general method for the synthesis of the peptidomimetics of the present invention is exemplified in the following paragraphs (Procedures A-D). This is to demonstrate the principal synthetic concept and does not limit or restrict the present invention in any way. A person skilled in the art is easily able to modify these procedures.

### Procedure A. Coupling of the First Protected Amino Acid Residue to the Resin

In a dried flask, 2-chlorotrityl chloride resin (polystyrene, 1% crosslinked; loading: 1.4 mMol/g) was swollen in dry DCM for 30 min (7 mL DCM per g resin). A solution of 0.8 eq of the Fmoc-protected amino acid and 6 eq of DIPEA in dry DCM/DMF (4/1) (10 mL per g resin) was added. After shaking for 2-4 h at rt the resin was filtered off and washed successively with DCM, DMF, DCM, DMF and DCM. Next, the resin was treated with a solution of dry DCM/MeOH/DIPEA (17:2:1) (10 mL per g resin) for 3×30 min. The resin was filtered off in a pre-weighed sinter funnel and washed successively with DCM, DMF, DCM, MeOH, DCM, MeOH, DCM (2×) and Et₂O (2×). The resin was dried under high vacuum overnight. The final mass of resin was calculated before qualitative control.

Loading was typically 0.6-0.7 mMol/g.

### Procedure B. Synthesis of the Fully Protected Peptide Fragment

The synthesis was carried out on a Symphony-X-peptide synthesizer (Gyros Protein Technologies) using 12 to 24 reaction vessels. Depending on the scale used (0.125 to 0.5 mmol), the above resin was placed into a reactor of appropriate size.

The following reaction cycles were programmed and carried out:

| Step | Reagent Time | | |
|---|---|---|---|
| 1 | DCM, wash and swell (start of synthesis only) | 1 × 3 | min |
| 2 | DMF, wash and swell | 3 × 10 | min |
| 3 | 20% piperidine/DMF | 2 × 2 | min |
| 4 | DMF, wash | 5 × 1 | min |
| 5 | 4 eq Fmoc amino acid in DMF + | 1 × 30 | min |
| | 4 eq HATU in DMF + | | |
| | 8 eq NMM in DMF | | |
| 6 | 4 eq Fmoc amino acid in DMF + | 1 × 30 | min |
| | 4 eq HATU in DMF + | | |
| | 8 eq NMM in DMF | | |
| 7 | DMF, wash | 2 × 1 | min |
| 8 | 16 eq acetic anhydride + | 1 × 5 | min |
| | 16 eq NMM | | |
| 9 | DMF, wash | 2 × 1 | min |
| 10 | 20% piperidine/DMF | 2 × 2 | min |
| 11 | DMF, wash | 5 × 1 | min |
| 12 | DCM, wash (at the end of the synthesis) | 3 × 1 | min |

Steps 5 to 10 are repeated to add each amino-acid residue. Fmoc/tBu/Trt/Pbf protected amino acids building blocks were used.

### Procedure C. Cleavage/Deprotection

After assembly of the protected peptide, the resin was suspended for 5 minute in the cleavage/deprotection cocktail TFA/dithiothreitol/water/TIS 85/102.5/2.5 v/v/v/v/v (7 mL/g of resin). After filtration, the resin was rinsed with a small volume of TFA. The combined filtrates were shaken for 3 h at room temperature. The linear peptide was precipitated in cold IPE and the obtained solid was washed three times with Et₂O. The white/off-white solid was air dried.

### Procedure D. Purification Procedure (Preparative Reverse Phase HPLC)

Compounds were purified by preparative reverse phase chromatography using two column Waters XBridge Prep C18 column, 50×150 mm, 5 μm in series.

Mobile phases used were:
A: 0.1% TFA in Water/Acetonitrile 98/2 v/v
B: 0.1% TFA in Acetonitrile

Gradient slopes in the preparative runs were adapted each time based on analytical LC-MS analysis of the crude product. As an example, a typical run was executed with a flow rate of 90 mL/min running a gradient as follows:

| T (min) | Flow (ml/min) | % B |
|---|---|---|
| 0 | 0 | 0 |
| 0.5 | 90 | 0 |
| 1 | 90 | 0 |
| 2.1 | 90 | 10 |
| 4 | 90 | 10 |
| 50 | 90 | 30 |
| 50.2 | 90 | 100 |
| 60.0 | 90 | 100 |

Detection: UV @ 220 nm and 254 nm

Fractions collected were pooled, concentrated under reduced vacuum and lyophilized.

### 1.2 Analytical Method

Analytical HPLC retention times (RT, in minutes) were determined on HPLC system: Thermo Scientific Ultimate 3000RS, MS: Thermo Scientific MSQ plus utilizing a Ascentis Express C8 column, 100×3 mm, 2.7 μm, with the following solvents A (H₂O+0.1% TFA) and B (CH₃CN+0.085% TFA) and the gradient was run at 55° C. as follows:

| T (min) | Flow (ml/min) | % B |
|---|---|---|
| 0 | 1.4 | 5 |
| 0.1 | 1.4 | 5 |
| 7 | 1.4 | 55 |
| 7.02 | 1.4 | 97 |
| 7.5 | 1.4 | 97 |
| 7.52 | 1.4 | 5 |
| 8.8 | 1.4 | 5 |

Detection: MS (ESI positive 60V profile mode) and UV @ 220 nm and 254 nm

### 1.3 Synthesis of Peptide Sequences

### Preparation of Examples 1-8

The protected peptide was synthesized from C- to N-terminus. The starting amino acid functionalized resin (obtained following Procedure A) used for the synthesis corresponds to P¹⁶ in Table 1. The protected linear peptide immobilized on resin (Resin-P¹⁶-P¹⁵-P¹⁴-P¹³-P¹²-P¹¹-P¹⁰-P⁹-P⁸-P⁷-P⁶-P⁵-P⁴-P³-P²-P¹) was synthesized following Procedure B. Subsequently, the resin was swollen in DMF and N,N′-bis-Boc-guanylpyrazole (5-10 eq) in DMF was added to the resin. The reaction was shaken overnight, and the resin was thoroughly washed with DMF and DCM.

Cleavage/deprotection of the modified peptide was performed as described in Procedure C. The deprotected linear peptide was dissolved in ammonium acetate buffer 1M at pH 6 containing 5% DMSO v/v (140 mL/mmol). The peptide solution was stirred 48 h in an opened flask at room temperature. The crude was purified according to procedure D. The compounds are obtained as TFA salt.

Preparation of acetate salt of compounds was executed as follow. The resin AG 1-X2 resin acetate form (loading=1 mmol/g) was poured in a column and washed with 10 times the volume of the resin of de-ionized water. The TFA salt of the peptide residue was dissolved in de-ionized water and transferred on the top of the resin (tap closed), the flask was rinsed twice to get 2 volumes of resin in total and the peptide was eluted slowly. Once this volume was eluted, the resin was washed with 1 volume of resin. In total, 3 fractions were collected and analyzed, then combined, and lyophilized to provide the desire peptide as acetate salt. Analytical data for each example are summarized in Table 1 (SEQ ID NOS 9, 11, 10, 13-15, 12, and 16, respectively).

### 1.4 Sequence Data

TABLE 1
Examples sequences and analytical data

| | | | | | | | | | | | | | | | | | RT in | [M + | |
|---|---|---|---|---|---|---|---|---|---|---|---|---|---|---|---|---|---|---|---|
| Example no | P1 | P2 | P3 | P4 | P5 | P6 | P7 | P8 | P9 | P10 | P11 | P12 | P13 | P14 | P15 | P16 | min | nH]ⁿ⁺/n | n |
| 1 | Gua-V | Hyp | I | Val(3OH) | Y | Pen | N | R | Dab | T | ^{D}Dab | K | C | Dab | R | Y | 2.84 | 672.3 | 3 |
| 2 | Gua-V | Hyp | I | Val(3OH) | Y | Pen | N | R | Dab | S | ^{D}Dab | K | C | Dab | R | Y | 2.82 | 667.7 | 3 |
| 3 | Gua-V | Hyp | I | Val(3OH) | Y | Pen | N | R | Dab | T | ^{D}Dab | K | C | Dab | R | Y | 2.88 | 663.4 | 3 |
| 4 | Gaa-V | Hyp | I | Val(3OH) | Y | Pen | N | R | Dab | T | ^{D}Dab | Dab | C | Dab | Dab | Y | 2.82 | 644.3 | 3 |
| 5 | Gua-V | Hyp | I | Val(3OH) | Y | Pen | N | R | Hyp | T | ^{D}Dab | Dab | C | Dab | Dab | Y | 2.85 | 648.3 | 3 |
| 6 | Gua-V | Hyp | I | Val(3OH) | Y | Pen | N | R | Hyp | S | ^{D}Dab | Dab | C | Dab | Dab | Y | 2.76 | 643.9 | 3 |
| 7 | Gua-V | Hyp | I | Val(3OH) | Y | Pen | N | R | P | S | ^{D}Dab | Dab | C | Dab | Dab | Y | 2.85 | 638.7 | 3 |
| 8 | Gua-V | Hyp | I | Val(3OH) | Y | Pen | N | R | Dab | T | ^{D}Dab | Dab | C | Dab | T | Y | 2.99 | 644.8 | 3 |

Notes to Table 1:
Abbreviations of amino acid/amino acid residue or derivatives thereof: see listing above
The sequences of Examples 1-8 have disulfide bridges between P⁶ and P¹³, as described above

### 2. In Vitro Biological Studies

### 2.1. Preparation of the Peptides

Lyophilized peptides were weighed on a Microbalance (Mettler MT5) and dissolved in sterile water to a final concentration of 1 mg/mL. Stock solutions were kept at +4° C., light protected.

### 2.2. Antimicrobial Activity of the Peptides

The selective antimicrobial activities of the peptides were determined in 96-well plates (Greiner, polystyrene) by the standard CLSI broth microdilution method (Clinical and Laboratory Standards Institute. Methods for Dilution Antimicrobial Susceptibility Tests for Bacteria That Grow Aerobically; Approved Standard—Ninth Edition. CLSI document M07-A9 (ISBN 1-56238-783-9 [Print]; ISBN 1-56238-784-7 [Electronic]). Clinical and Laboratory Standards Institute, 950 West Valley Road, Suite 2500, Wayne, Pennsylvania 19087, USA, 2012) with slight modifications.

Colonies of the microorganisms were diluted in saline (0.85%, NaCl) and adjusted using a McFarland reader (bioMérieux S A, Marcy-l'Etoile, France) to 0.5 McFarland standard. Subsequently, the bacterial suspension was diluted in Mueller-Hinton II (MHII, cation adjusted) broth to give approximately 5×10⁵ colony forming units (CFU/mL).

Inocula of the microorganisms were diluted into Mueller-Hinton II (MH, cation adjusted) broth and compared with a 0.5 McFarland standard to give appr. 10⁶ colony forming units (CFU)/mL. Aliquots (90 μl) of inoculate were added to 10 μl of water+P-80 (Polysorbate 80, 0.002% final concentration) containing the peptide in serial two-fold dilutions at 10 fold final concentration. The following microorganisms were used to determine antibiotic selectivity of the peptides: Escherichia coli ATCC 25922, Escherichia coli MCR-1 Af 45, Klebsiella pneumoniae SSI3010, Klebsiella pneumoniae NCTC 13443 and Klebsiella pneumoniae ATCC 43816. Antimicrobial activities of the peptides were expressed as the minimal inhibitory concentration (MIC) in μg/mL at which no visible growth was observed after 18-20 hours of incubation at 35° C.

The major mechanism of spontaneous resistance to thanatin, leading to a substantial MIC increase involves the formation of the LptA Q62L mutant. It has been shown that frequency of resistance can be reduced by optimizing binding affinity for LptA Q62L. (Schuster et al., Sci. Adv. 9, eadg3683 (2023)). Q62L mutants of the parent strains were generated as described by Schuster et al. and the compounds were tested for MIC using the protocol described above.

### 2.3. Hemolysis

The peptides were tested for their hemolytic activity against human red blood cells (hRBC). Fresh hRBC were washed three times with phosphate buffered saline (PBS) and centrifuged for 5 min at 3000×g. Compounds (200 μg/mL) were incubated with 20% hRBC (v/v) for 1 h at 37° C. and shaking at 300 rpm. A value of 0% and 100% cell lyses, respectively, was determined by incubation of hRBC in the presence of PBS and 2.5% Triton X-100 in H₂O, respectively. The samples were centrifuged, the supernatants were 8-fold diluted in PBS buffer and the optical densities (OD) were measured at 540 nm. The 100% lyses value (OD₅₄₀H₂O) gave an OD₅₄₀ of approximately 0.5-1.0.

Percent hemolysis was calculated as follows: (OD₅₄₀peptide/OD₅₄₀H₂O)×100%.

The results of the experiments described in 2.2-2.3 are indicated in Table 2 and Table 3 herein below:

TABLE 2
Minimal inhibitory concentrations (MIC)
in Mueller-Hinton broth II and Hemolysis

| | Escherichia coli | Escherichia | Klebsiella | Hemolysis |
|---|---|---|---|---|
| Exam- | ATCC 25922 MIC | coli | pneumoniae | at 0.2 g/L |
| ple no | [mg/L] | MCR-1 Af 45 | SSI3010 | [%] |
| 1 | 0.0625 | 0.0625 | 0.125 | ND |
| 2 | 0.03125 | 0.03125 | 0.0625 | ND |
| 3 | 0.0625 | 0.0625 | 0.125 | 0 |
| 4 | 0.03125 | 0.03125 | 0.0625 | 0 |
| 5 | 0.25 | 0.25 | 0.25 | 0 |
| 6 | 0.125 | 0.25 | 0.25 | 0 |
| 7 | 0.25 | 0.09375 | 0.25 | 0 |
| 8 | 0.0625 | 0.0625 | 0.125 | 0 |

Note to Table 2:
ND = Not determined

TABLE 3
Minimal inhibitory concentrations (MIC) in Mueller-Hinton broth II

| | Escherichia coli ATCC | | Klebsiella pneumoniae | | Klebsiella pneumoniae | |
|---|---|---|---|---|---|---|
| | 25922 | | ATCC 43816 | | NCTC 13443 | |
| | MIC [mg/L] | | MIC [mg/L] | | MIC [mg/L] | |
| | | Q62L | | Q62L | | Q62L |
| Example no | Parent | mutant | Parent | mutant | Parent | mutant |
| 1 | 0.0625 | 0.5 | 0.25 | 2 | 1 | 4 |
| 2 | 0.03125 | 0.375 | 0.125 | 0.75 | 0.375 | 3 |
| 3 | 0.0625 | 0.5 | 0.25 | 2 | 0.5 | 4 |
| 4 | 0.03125 | 0.25 | 0.0625 | 0.5 | 0.25 | 2 |
| 5 | 0.25 | 1 | 0.5 | 1 | 1 | 2 |
| 6 | 0.125 | 1 | 0.5 | 1 | 1 | 2 |
| 7 | 0.25 | 0.5 | 0.375 | 1.5 | 1 | 3 |
| 8 | 0.0625 | 0.25 | 0.25 | 1 | 1 | 2 |

### 3. In Vivo Biological Studies

### 3.1 Methods

### 3.1.1 Animal Preparation

The lung infection models with K. pneumoniae ATCC 43816 strains were performed with neutropenic female ICR mice, weighing 22±2 g. All animals were specific pathogen-free. The animals were made neutropenic by two intraperitoneal (IP) injections of cyclophosphamide (CP) following a standard method for mouse lung infection models (Zuluaga A F et. al., BMC Infectious Diseases 2006, 6, 55). The first dose of CP was given at 150 mg/kg 4 days before infection (Day −4), and the second at 100 mg/kg 1 day before infection (Day −1), prior to infection on Day 0. It was determined in prior complete blood count studies that this cyclophosphamide treatment schedule resulted in neutropenia (<100 neutrophils per μL) until Day 2 after infection (data not shown).

### 3.1.2 Inoculum Preparation

A 0.2 mL aliquot of a single-use glycerol stock (at −80° C.) of K. pneumoniae culture was used to seed 20 mL brain heart infusion broth (BHI) (237500, BD, USA) and then incubated at 35-37° C. with shaking (120 rpm) for 8 h. Bacterial cells in 20 mL culture were pelleted by centrifugation (3,500×g) for 15 minutes, then re-suspended in 10 mL cold Phosphate buffer saline (PBS) (P4417, Sigma, USA). The optical density, OD_{620 nm}, was measured and used to guide dilutions to achieve the target bacterial density. The bacteria were further diluted with BHI and stored on ice for no more than one hour prior to animal inoculation. Bacterial counts in the challenge organism suspension were enumerated by dilution plating to nutrient agar (NA) plates.

### 3.1.3 Challenge

On Day 0, animals were infected with bacterial suspension by intranasal injection. Animals were deeply anesthetized with pentobarbital sodium (50 mg/kg, IV). The mouse was held upright and then drops of bacterial suspension were gradually released into the nares using a micropipette until a total volume of 20 μL/mouse, 10 μL per nostril was inoculated. The mouse was held in an upright position for a couple of minutes until the breath returned to a normal rate and depth then placed into the cage for recovery. In the K. pneumoniae ATCC 43816 lung infection model the target inoculum size was 1×10⁵ CFU/mouse, and the actual inoculum sizes were 1.26×10⁵, and 1.25×10⁵ CFU/mouse for efficacy assays for Example 4, and Example 8, respectively. In the K. pneumoniae NCTC13443 lung infection model the target inoculum size was 0.5-1×10⁶ CFU/mouse, and the actual inoculum sizes were 5.6×10⁵ CFU/mouse.

### 3.1.4 Treatment

Test articles, Example 4 and Example 8 and vehicle were administered to animals by IV slow bolus (30 s) administration using the dosing schedule, volumes, and concentrations indicated below. Dosing solutions were prepared in 0.9% NaCl and the pH was adjusted to pH 6-7. Animals were observed at 5 min after dosing to detect acute toxicity, which was to be recorded and reported if observed. Reference control agents, were administered as indicated below.

### 3.1.5 Post Challenge

Animal observations and humane endpoints. Animals were checked for humane endpoints at 12 h after infection. Animals were to be humanely sacrificed if found in distress or a moribund state. Euthanasia was performed following the 2020 AVMA Guidelines on Euthanasia. Animals were euthanized using compressed CO₂ gas in a CO₂ gas chamber. Tissues were not recovered if animals were euthanized prior to the scheduled sacrifice time point.

Tissue harvest. Animals were sacrificed with CO₂ asphyxiation at the scheduled time points for tissue harvest, at 2 or 26 h after infection. For lung-infected animals, lung tissue was aseptically harvested from each of the sacrificed animals, weighed, and homogenized in 1 mL sterile PBS (pH 7.4) with a Polytron homogenizer. Bacterial burden in the tissue homogenates was determined by plating 10-fold serial dilutions and plating 0.1 mL of each to MacConkey II agar plates (A01-41, CMP, Taiwan). The CFU per tissue (CFU/tissue) was calculated.

### 3.1.6 Data Analysis and Statistics

For each animal, the following raw data were recorded and tabulated: tissue weight and bacterial counts in each tissue homogenate dilution. For each tissue, the homogenate dilution that yielded the largest number of colonies, between 10 to 300 colonies per plate, was selected to calculate the bacterial counts per tissue (CFU/tissue). Bacterial counts per tissue (CFU/tissue) were tabulated and plotted in GraphPad® Prism. The raw colony count data of the homogenate dilutions was inspected for proportionality within the dilution series. The 10-fold serial dilutions are expected to show 10-fold reductions in counts. Disproportionate data, such as fewer counts in the undiluted homogenate samples compared to the diluted sample, would indicate inhibition of colony growth due to drug carryover from the tissue to the test plate. No aberrant titration data were observed.

The average bacterial counts in tissues (CFU/tissue) were calculated for each treatment group. Data were plotted in GraphPad® Prism as the bacterial counts per tissue of control and treatment groups. The difference in bacterial density between the baseline group (2 h initial counts) and the treatment group was calculated. Significance was assessed with ANOVA analysis using GraphPad® Prism software.

Δ=log ₁₀(CFU/tissue of 26 h)−log ₁₀(CFU/tissue of baseline)

Nonlinear regression was conducted to estimate the doses that result in bacteriostasis, 1-log ₁₀, and 2-log ₁₀ reductions in bacterial counts. The estimated EC₅₀ for each model was determined if available.

### 3.1.7 Data Interpretation

The dose levels that resulted in bacteriostasis, with no net increase in counts relative to baseline at 2 h after infection were considered to be efficacious. The dose levels that result in 1-log ₁₀ and 2-log ₁₀ reductions in counts relative to baseline are a substantial bacterial killing effect.

### 3.1.7 Results

3.2.1 K. pneumoniae ATCC 43816 Lung Infection Model

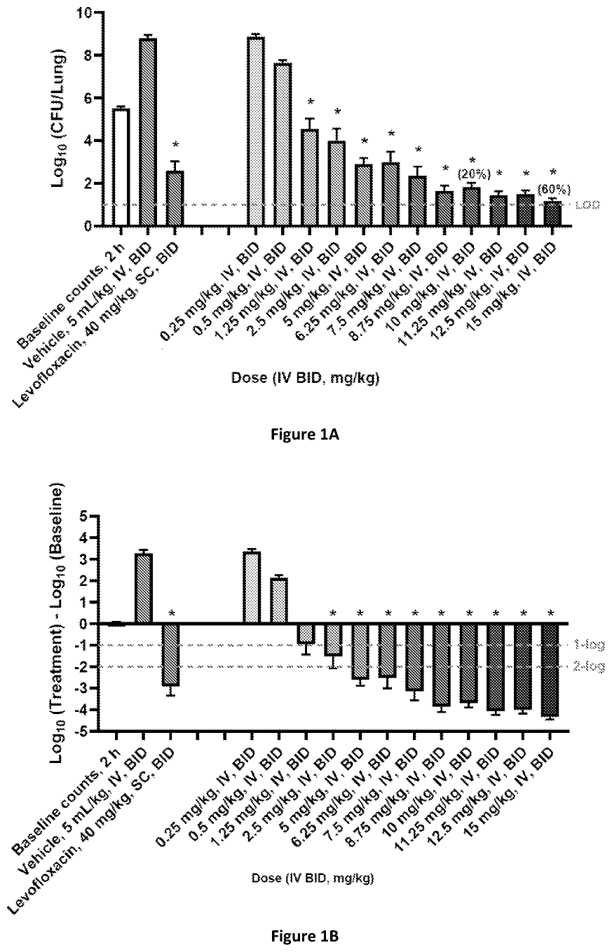

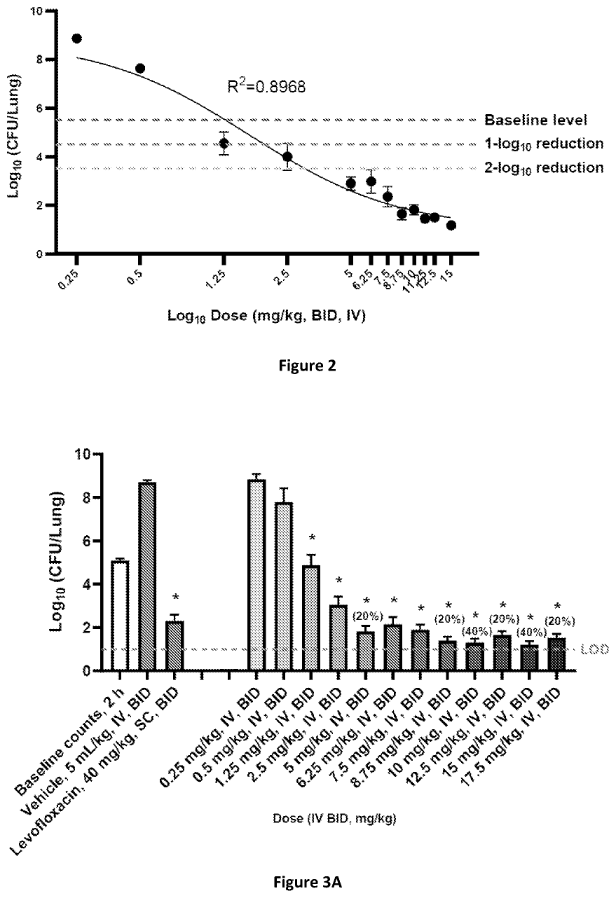

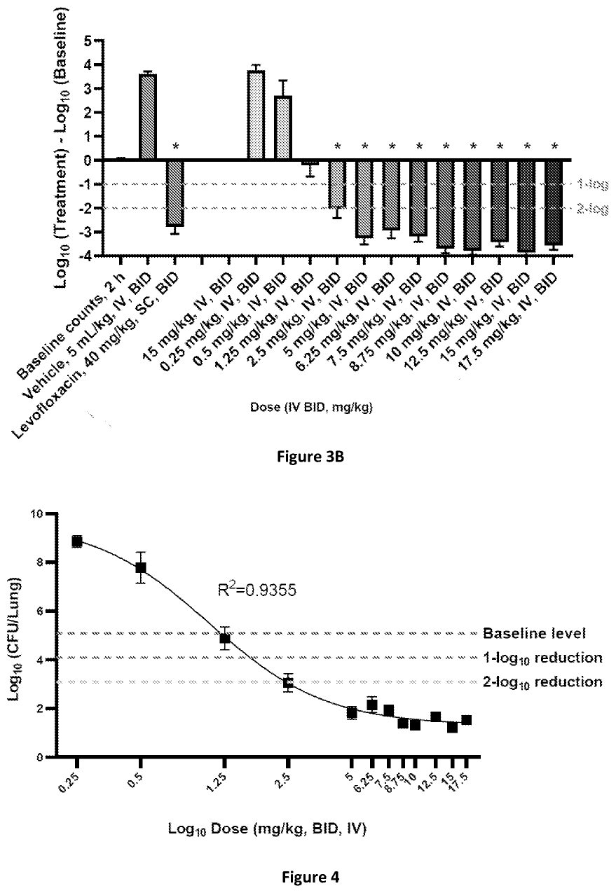

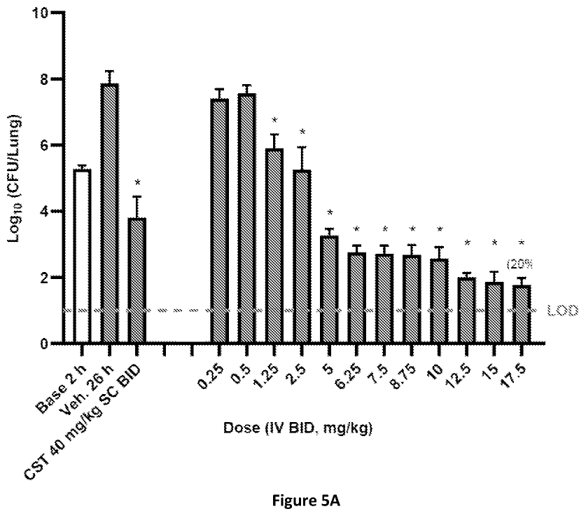

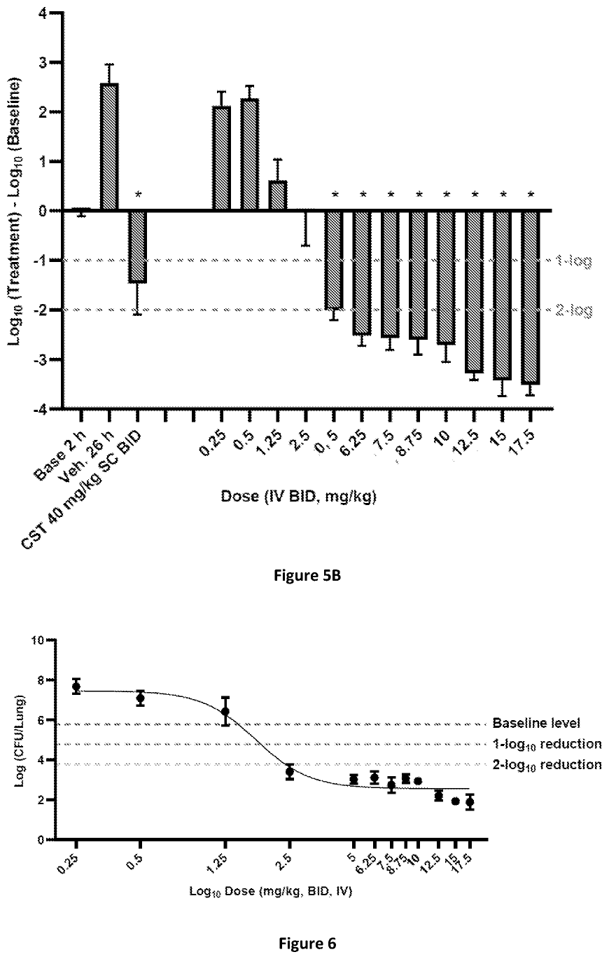

The efficacy of Example 4 was evaluated in the K. pneumoniae ATCC 43816 lung infection model with neutropenic mice. Strain K. pneumoniae ATCC 43816 grew well and resulted in a 3.3-log ₁₀ increase in bacterial counts at 26 h post-infection (vehicle group with 8.8-log ₁₀ CFU/lung) relative to the baseline count (2 h post-infection group with 5.5-log ₁₀ CFU/lung). The control reference agent, levofloxacin at 40 mg/kg, was subcutaneously (SC) administered twice (BID) with a 12 h interval (q12 h) at 2 and 14 h after infection and resulted in a 2.9-log ₁₀ reduction in bacterial counts compared to the baseline count (p<0.05) (FIGS. 1A and 1B).

Example 4 at 12 doses ranging from 0.25 to 15 mg/kg was IV slow bolus (30 s) administered BID q12 h at 2 and 14 h after infection. No acute toxicity side effects were observed in this efficacy study. A dose-responsive effect was observed in the Example 4 treatment groups compared to the baseline group. Significant 1.5- to 4.3-log ₁₀ reductions in bacterial counts (p<0.05) were found at 2.5, 5, 6.25, 7.5, 8.75, 10, 11.25, 12.5, and 15 mg/kg IV, BID q12 h, in comparison to the baseline control group (FIGS. 1A and 1B). An EC₅₀ value of 1.65 mg/kg IV q12 h and a Hillslope value of −1.220 were estimated using nonlinear regression with the Hill equation. The estimated doses that achieved bacteriostasis, 1-log ₁₀, and 2-log ₁₀ kill values were 1.08, 1.81, and 3.18 mg/kg IV q12 h, respectively (FIG. 2). At a lower concentration of 1.25 mg/kg IV BID q12 h, administration of Example 4 did not achieve bacteriostasis but resulted in significant reductions in bacterial counts (p<0.05) compared to the vehicle control (FIG. 1A).

The efficacy of Example 8 was evaluated in the K. pneumoniae ATCC 43816 lung infection model with neutropenic mice. Strain K. pneumoniae ATCC 43816 grew well and resulted in a 3.6-log ₁₀ increase in bacterial counts at 26 h post-infection (vehicle group, at 8.7-log ₁₀ CFU/lung) relative to the baseline count (2 h post-infection group, at 5.1-log ₁₀ CFU/lung). The control reference agent, levofloxacin at 40 mg/kg, was subcutaneously (SC) administered twice (BID) with a 12 h interval (q12 h) at 2 and 14 h after infection and resulted in a 2.8-log ₁₀ reduction in bacterial counts compared to the baseline count (p<0.05) (FIGS. 3A and 3B).

Example 8 at 12 doses ranging from 0.25 to 17.5 mg/kg, was IV slow bolus (30 s) administered BID q12 h at 2 and 14 h after infection. No acute toxicity side effects were observed in this efficacy study. A dose-responsive effect was observed in the Example 8 treatment groups compared to the baseline group. Significant 2.0- to 3.9-log ₁₀ reductions in bacterial counts (p<0.05) were found at 2.5, 5, 6.25, 7.5, 8.75, 10, 12.5, 15, and 17.5 mg/kg IV, BID q12 h, in comparison to the baseline control group (FIGS. 3A and 3B). An EC₅₀ value of 1.06 mg/kg IV q12 h and a Hillslope value of −1.571 were estimated using nonlinear regression with the Hill equation. The estimated doses that achieved bacteriostasis, 1-log ₁₀, and 2-log ₁₀ kill values were 1.20, 1.65, and 2.44 mg/kg IV q12 h, respectively (FIG. 4). At a lower concentration of 1.25 mg/kg IV BID q12 h, administration of Example 8 did not achieve bacteriostasis but resulted in significant reductions in bacterial counts (p<0.05) compared to the vehicle control (FIG. 3A).

3.2.2 K. pneumoniae NCTC13443 Lung Infection Model

The efficacy of Example 8 was evaluated in the K. pneumoniae NCTC 13443 lung infection model with neutropenic ICR female mice. Strain K. pneumoniae NCTC 13443 grew well and resulted in a 2.58-log ₁₀ increase in bacterial counts at 26 h post-infection (vehicle group with 7.86-log ₁₀ CFU/lung) relative to the baseline count (2 h post-infection group with 5.28-log ₁₀ CFU/lung). The control reference agent, colistin at 40 mg/kg, was subcutaneously (SC) administered twice (BID) with a 12 h interval (q12 h) at 2 and 14 h after infection and resulted in a 1.47-log ₁₀ reduction in bacterial counts compared to the baseline count (p<0.05) (FIGS. 5A and 5B).

Example 8 at 12 doses ranging from 0.25 to 17.5 mg/kg were IV slow bolus (30 s) administered BID q12 h at 2 and 14 h after infection. No acute toxicity side effects were observed in this efficacy study. A dose-responsive effect was observed in the Example 8 treatment group compared to the baseline group. Significant 2.01- to 3.51-log ₁₀ reductions in bacterial counts (p<0.05) were found at 5, 6.25, 7.5, 8.75, 10, 12.5, 15, and 17.5 mg/kg IV BID q12 h, in comparison to the baseline control group (FIGS. 5A and 5B). An EC₅₀ value of 1.74 mg/kg IV BID q12 h and a Hillslope value of −3.448 were estimated using nonlinear regression with the Hill equation. The estimated doses that achieved bacteriostasis, 1-log ₁₀, and 2-log ₁₀ kill values were 1.63, 2.07, and 2.89 mg/kg IV BID q12 h, respectively (FIG. 6). Two lower concentrations of 2.5 and 1.25 mg/kg IV BID q12 h both resulted in significant reductions in bacterial counts (p<0.05) compared to the vehicle control and the 2.5 mg/kg dose achieved the bacteriostasis compared to the baseline count (FIGS. 5A and 5B). No significant bacterial counts reductions were found at two other concentrations at 0.25, and 0.5 mg/kg IV BID q12 h compared to the vehicle control (FIG. 5A).

1. A compound of the general formula (I),
```
  (SEQ ID NO: 17)
P¹-P²-P³-P⁴-P⁵-P⁶-P⁷-P⁸-P⁹-P¹⁰-P¹¹-P¹²-P¹³-P¹⁴-P¹⁵-P¹⁶
                                                          (I)
```
wherein
P¹ is Gua-Val;
P² is Pro, Pro(4R)OMe, Pro(3,4dehydro), Pic, Pro((4R)NH₂), Ndab, NalloThr, or Hyp;
P³ is Ile;
P⁴ is Val(3OH);
P⁵ is Tyr;
P⁶ is Cys or Pen;
P⁷ is Asn, Leu, Ile, Ser, Dap, or His;
P⁸ is Arg;
P⁹ is Arg, Lys, Dab, Dab(iPr), Pro or Hyp;
P¹⁰ is Ser or Thr;
P¹¹ is ^{D}Dab, ^{D}Dab(iPr), or ^{D}Arg;
P¹² is Lys, Ile, Ser, Dab, Orn, or Cit;
P¹³ is Cys or Pen;
P¹⁴ is Dab, Dab(iPr), Lys, Gln, Ser, or Tyr;
P¹⁵ is Arg, Dab, Orn, Orn (iPr), Ser, or Thr;
P¹⁶ is Nle, Cha, or Tyr;
or a salt thereof,
wherein Cys or Pen at P⁶ and Cys or Pen at P¹³ optionally form a disulfide bridge between P⁶ and P¹³;
with the proviso that at least three amino acid residues among the four amino acid residues at positions P⁹, P¹², P¹⁴ and P¹⁵ are basic amino acid residues selected from Dab, Lys, Arg, or Dab(iPr) at P⁹, Lys, Orn, or Dab at P¹², Lys, Dab or Dab(iPr) at P¹⁴ and Arg, Dab, Orn, or Orn (iPr) at P¹⁵.

2. The compound according claim 1, wherein
P¹ is Gua-Val;
P² is Pro, Pro(4R)OMe, Pro(3,4dehydro), Pic, Pro((4R)NH₂), Ndab, NalloThr, or Hyp;
P³ is Ile;
P⁴ is Val(3OH);
P⁵ is Tyr;
P⁶ is Cys or Pen;
P⁷ is Asn, Leu, Ile, Ser, Dap, or His;
P⁸ is Arg;
P⁹ is Arg, Lys, Dab, Dab(iPr), Pro or Hyp;
P¹⁰ is Ser or Thr;
P¹¹ is ^{D}Dab, ^{D}Dab(iPr), or ^{D}Arg;
P¹² is Dab;
P¹³ is Cys or Pen;
P¹⁴ is Dab, Dab(iPr), Lys, Gln, Ser, or Tyr;
P¹⁵ is Arg, Dab, Orn, Orn (iPr), Ser, or Thr;
P¹⁶ is Nle, Cha, or Tyr;
or a salt thereof,
wherein Cys or Pen at P⁶ and Cys or Pen at P¹³ optionally form a disulfide bridge between P⁶ and P¹³;
with the proviso that at least two amino acid residues among the three amino acid residues at positions P⁹, P¹⁴ and P¹⁵ are basic amino acid residues selected from Dab, Lys, Arg, or Dab(iPr) at P⁹, Lys, Dab or Dab(iPr) at P¹⁴ and Arg, Dab, Orn, or Orn (iPr) at P¹⁵.

3. The compound according to claim 1, wherein
P¹ is Gua-Val;
P² is Pro, Pro(4R)OMe, Pro(4R)NH₂), or Hyp;
P³ is Ile;
P⁴ is Val(3OH);
P⁵ is Tyr;
P⁶ is Cys or Pen;
P⁷ is Asn;
P⁸ is Arg;
P⁹ is Lys, Dab, Dab(iPr), Pro or Hyp;
P¹⁰ is Ser or Thr;
P¹¹ is ^{D}Dab, ^{D}Dab(iPr);
P¹² is Dab;
P¹³ is Cys or Pen;
P¹⁴ is Dab, Dab(iPr), Lys;
P¹⁵ is Arg, Dab, Orn, Orn (iPr), Ser, or Thr;
P¹⁶ is Tyr;
or a salt thereof,
wherein Cys or Pen at P⁶ and Cys or Pen at P¹³ optionally form a disulfide bridge between P⁶ and P¹³;
with the proviso that at least one amino acid residue among the two amino acid residues at positions P⁹ and P¹⁵ are basic amino acid residues selected from Dab, Lys or Dab(iPr) at P⁹, and Arg, Dab, Orn, or Orn (iPr) at P¹⁵.

4. The compound according to claim 1, wherein
P¹ is Gua-Val;
P² is Hyp;
P³ is Ile;
P⁴ is Val(3OH);
P⁵ is Tyr;
P⁶ is Pen;
P⁷ is Asn or Ser;
P⁸ is Arg;
P⁹ is Dab, Pro or Hyp;
P¹⁰ is Ser or Thr;
P¹¹ is ^{D}Dab;
P¹² is Lys or Dab;
P¹³ is Cys;
P¹⁴ is Dab;
P¹⁵ is Arg, Dab or Thr;
P¹⁶ is Tyr;
or a salt thereof,
wherein Pen at P⁶ and Cys at P¹³ optionally form a disulfide bridge between P⁶ and P¹³;
with the proviso that at least one amino acid residue among the two amino acid residues at positions P⁹ and P¹⁵ are basic amino acid residues selected from Dab at P⁹, and Arg and Dab at P¹⁵.

5. The compound according to claim 1, wherein
P¹ is Gua-Val;
P² is Hyp;
P³ is Ile;
P⁴ is Val(3OH);
P⁵ is Tyr;
P⁶ is Pen;
P⁷ is Asn;
P⁸ is Arg;
P⁹ is Dab, Pro or Hyp;
P¹⁰ is Ser or Thr;
P¹¹ is ^{D}Dab;
P¹² is Dab;
P¹³ is Cys;
P¹⁴ is Dab;
P¹⁵ is Dab or Thr;
P¹⁶ is Tyr;
or a salt thereof,
wherein Pen at P⁶ and Cys at P¹³ optionally form a disulfide bridge between P⁶ and P¹³;
with the proviso that at least one amino acid residue among the two amino acid residues at positions P⁹ and P¹⁵ are basic amino acid residues selected from Dab at P⁹, and Dab at P¹⁵.

6. The compound according to claim 1, wherein the compound is selected from the group consisting of:
(SEQ ID NO: 1)
Gua-Val-Hyp-Ile-Val(3OH)-Tyr-Pen-Asn-Arg-Dab-Thr-
^{D}Dab-Lys-Cys-Dab-Arg-Tyr;
(SEQ ID NO: 2)
Gua-Val-Hyp-Ile-Val(3OH)-Tyr-Pen-Ser-Arg-Dab-Thr-
^{D}Dab-Lys-Cys-Dab-Arg-Tyr;
(SEQ ID NO: 3)
Gua-Val-Hyp-Ile-Val(3OH)-Tyr-Pen-Asn-Arg-Dab-Ser-
^{D}Dab-Lys-Cys-Dab-Arg-Tyr;
(SEQ ID NO: 4)
Gua-Val-Hyp-Ile-Val(3OH)-Tyr-Pen-Asn-Arg-Pro-Ser-
^{D}Dab-Dab-Cys-Dab-Dab-Tyr;
(SEQ ID NO: 5)
Gua-Val-Hyp-Ile-Val(3OH)-Tyr-Pen-Asn-Arg-Dab-Thr-
^{D}ab-Dab-Cys-Dab-Dab-Tyr;
(SEQ ID NO: 6)
Gua-Val-Hyp-Ile-Val(3OH)-Tyr-Pen-Asn-Arg-Hyp-Thr-
^{D}Dab-Dab-Cys-Dab-Dab-Tyr;
(SEQ ID NO: 7)
Gua-Val-Hyp-Ile-Val(3OH)-Tyr-Pen-Asn-Arg-Hyp-Ser-
DDab-Dab-Cys-Dab-Dab-Tyr;
and
(SEQ ID NO: 8)
Gua-Val-Hyp-Ile-Val(3OH)-Tyr-Pen-Asn-Arg-Dab-Thr-
^{D}Dab-Dab-Cys-Dab-Thr-Tyr;
or a salt thereof,
wherein Pen at P⁶ and Cys at P¹³ optionally form a disulfide bridge between P⁶ and P¹³.

7. The compound according to claim 1, wherein the compound is selected from the group consisting of:
(SEQ ID NO: 4)
Gua-Val-Hyp-Ile-Val(3OH)-Tyr-Pen-Asn-Arg-Pro-Ser-
^{D}Dab-Dab-Cys-Dab-Dab-Tyr;
(SEQ ID NO: 5)
Gua-Val-Hyp-Ile-Val(3OH)-Tyr-Pen-Asn-Arg-Dab-Thr-
^{D}Dab-Dab-Cys-Dab-Dab-Tyr;
(SEQ ID NO: 6)
Gua-Val-Hyp-Ile-Val(3OH)-Tyr-Pen-Asn-Arg-Hyp-Thr-
^{D}Dab-Dab-Cys-Dab-Dab-Tyr;
(SEQ ID NO: 7)
Gua-Val-Hyp-Ile-Val(3OH)-Tyr-Pen-Asn-Arg-Hyp-Ser-
^{D}Dab-Dab-Cys-Dab-Dab-Tyr;
and
(SEQ ID NO: 8)
Gua-Val-Hyp-Ile-Val(3OH)-Tyr-Pen-Asn-Arg-Dab-Thr-
^{D}Dab-Dab-Cys-Dab-Thr-Tyr;
a salt thereof;
wherein Pen at P⁶ and Cys at P¹³ optionally form a disulfide bridge between P⁶ and P¹³.

8. The compound according to any one of claims 1 to 7, wherein Pen at P⁶ and Cys at P¹³ form a disulfide bridge between P⁶ and P¹³.

9. The compound according to any one of claims 1 to 8, wherein the compound disrupts the lipopolysaccharide transport protein bridge in Gram-negative bacteria.

10. An enantiomer of a compound of formula (I) as defined in any one of claims 1 to 9.

11. A pharmaceutical composition containing a compound of formula (I) or a pharmaceutically acceptable salt thereof or a mixture of compounds of formula (I) or pharmaceutically acceptable salts thereof as defined in any one of claims 1 to 9 and at least one pharmaceutically inert carrier.

12. A compound of formula (I) according to any one of claims 1 to 9, or a pharmaceutically acceptable salt thereof, for use as a pharmaceutically active substance having antibiotic activity.

13. Use of a compound of formula (I) according to any one of claims 1 to 9, or a pharmaceutically acceptable salt thereof, as a pharmaceutically active substance having antibiotic activity.

14. A compound according to any one of claims 1 to 9, or a pharmaceutically acceptable salt thereof, for use as a medicament.

15. The compound for use according to claim 14, for use in a method of treating or preventing a bacterial infection or a disease related to such infection.

16. The compound for use according to claim 14 or claim 15, wherein the infection is related to respiratory diseases or skin or soft tissue diseases or gastrointestinal diseases or eye diseases or ear diseases or CNS diseases or bone diseases or cardiovascular diseases or genitourinary diseases, or nosocomial infections, or catheter-related and non-catheter-related infections, or urinary tract infections, or bloodstream infections; or infection-induced sepsis.

17. The compound for use according to any one of claims 14 to 16, wherein the bacterial infection or disease related to such infection is caused by Gram-negative bacteria.

18. The compound according to claim 17, wherein the Gram-negative bacteria is Enterobacteriaceae.

19. The compound according to claim 18, wherein the Enterobacteriaceae is Klebsiella pneumoniae and/or Escherichia coli.

20. Use of a compound according to any one of claims 1 to 9, or a salt thereof, as a disinfectant or preservative for foodstuffs, cosmetics, medicaments, and/or other nutrient-containing materials.

21. A method of treating or preventing a bacterial infection or a disease related to such infection in a subject in need thereof, comprising administering a therapeutically effective amount of a compound of formula (I) as defined in any one of claim 1 to 9, or a pharmaceutically acceptable salt thereof, to the subject.

22. Use of a compound of formula (I) as defined in any one of claims 1 to 9, or a pharmaceutically acceptable salt thereof, in the manufacture of a medicament for the treatment or prevention of a bacterial infection or a disease related to such infection.


SEQUENCE LISTING (17 sequences)

SEQ ID NO: 1 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  VARIANT 6..13: note = Optional intrachain disulfide bond
VXIVYXNRXTXKCXRY

SEQ ID NO: 2 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  VARIANT 6..13: note = Optional intrachain disulfide bond
VXIVYXSRXTXKCXRY

SEQ ID NO: 3 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  VARIANT 6..13: note = Optional intrachain disulfide bond
VXIVYXNRXSXKCXRY

SEQ ID NO: 4 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  VARIANT 6..13: note = Optional intrachain disulfide bond
VXIVYXNRPSXXCXXY

SEQ ID NO: 5 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  VARIANT 6..13: note = Optional intrachain disulfide bond
VXIVYXNRXTXXCXXY

SEQ ID NO: 6 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (2S,4R)-4-hydroxypyrrolidine-2-carboxylic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  VARIANT 6..13: note = Optional intrachain disulfide bond
VXIVYXNRXTXXCXXY

SEQ ID NO: 7 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (2S,4R)-4-hydroxypyrrolidine-2-carboxylic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  VARIANT 6..13: note = Optional intrachain disulfide bond
VXIVYXNRXSXXCXXY

SEQ ID NO: 8 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  VARIANT 6..13: note = Optional intrachain disulfide bond
VXIVYXNRXTXXCXTY

SEQ ID NO: 9 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  SITE 6..13: note = Intrachain disulfide bond
VXIVYXNRXTXKCXRY

SEQ ID NO: 10 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  SITE 6..13: note = Intrachain disulfide bond
VXIVYXSRXTXKCXRY

SEQ ID NO: 11 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 14: note = (S)-2,4-diaminobutanoic acid
  SITE 6..13: note = Intrachain disulfide bond
VXIVYXNRXSXKCXRY

SEQ ID NO: 12 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  SITE 6..13: note = Intrachain disulfide bond
VXIVYXNRPSXXCXXY

SEQ ID NO: 13 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  SITE 6..13: note = Intrachain disulfide bond
VXIVYXNRXTXXCXXY

SEQ ID NO: 14 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (2S,4R)-4-hydroxypyrrolidine-2-carboxylic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  SITE 6..13: note = Intrachain disulfide bond
VXIVYXNRXTXXCXXY

SEQ ID NO: 15 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (2S,4R)-4-hydroxypyrrolidine-2-carboxylic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 15: note = (S)-2,4-diaminobutanoic acid
  SITE 6..13: note = Intrachain disulfide bond
VXIVYXNRXSXXCXXY

SEQ ID NO: 16 - protein, 16 aa, synthetic construct
  SITE 1: note = N-amidino-L-valine
  SITE 2: note = (2S,4R)-4-hydroxypyrrolidine-2-carboxylic acid
  SITE 4: note = 3-Hydroxy-L-valine
  SITE 6: note = (R)-2-amino-3-mercapto-3-methylbutanoic acid
  SITE 9: note = (S)-2,4-diaminobutanoic acid
  SITE 11: note = (R)-2,4-diaminobutanoic acid
  SITE 12: note = (S)-2,4-diaminobutanoic acid
  SITE 14: note = (S)-2,4-diaminobutanoic acid
  SITE 6..13: note = Intrachain disulfide bond
VXIVYXNRXTXXCXTY

SEQ ID NO: 17 - protein, 16 aa, synthetic construct
  SITE 1: note = N-amidino-L-valine
  VARIANT 2: note = Pro, (2S,4R)-4-methoxypyrrolidine-2-carboxylic acid, (S)-2,5-dihydro-1H-pyrrole-2-carboxylic acid, (S)-piperidine-2-carboxylic acid, (2S,4R)-4-aminopyrrolidine-2-carboxylic acid, N-(2-aminoethyl)glycine, N-((S)-Hydroxyethyl)glycine, or (2S,4R)-4-hydroxypyrrolidine-2-carboxylic acid
  SITE 4: note = 3-Hydroxy-L-valine
  VARIANT 6: note = Cys or (R)-2-amino-3-mercapto-3-methylbutanoic acid
  VARIANT 7: note = Asn, Leu, Ile, Ser, (5)-2,3-diaminopropanoic acid, or His
  VARIANT 9: note = Arg, Lys, (S)-2,4-diaminobutanoic acid, (S)-2-amino-4-(isopropylamino)butanoic acid, Pro or (2S,4R)-4-hydroxypyrrolidine-2-carboxylic acid
  VARIANT 10: note = Ser or Thr
  VARIANT 11: note = (R)-2,4-diaminobutanoic acid, (R)-2-amino-4-(isopropylamino)butanoic acid, or D-Arginine
  VARIANT 12: note = Lys, Ile, Ser, (S)-2,4-diaminobutanoic acid, (S)-2,5-diaminopentanoic acid, or (S)-2-amino-5-ureidopentanoic acid
  VARIANT 13: note = Cys or (R)-2-amino-3-mercapto-3-methylbutanoic acid
  VARIANT 14: note = (S)-2,4-diaminobutanoic acid, (S)-2-amino-4-(isopropylamino)butanoic acid, Lys, Gln, Ser, or Tyr
  VARIANT 15: note = Arg, (S)-2,4-diaminobutanoic acid, (S)-2,5-diaminopentanoic acid, (S)-2-amino-5-(isopropylamino)pentanoic acid, Ser, or Thr
  VARIANT 16: note = (S)-2-aminohexanoic acid, (S)-2-amino-3-cyclohexylpropanoic acid, or Tyr
  VARIANT 6..13: note = Optional intrachain disulfide bond
VXIVYXXRXXXXXXXX